SEQUENCE LISTING (83 sequences)

SEQ ID NO: 1 - protein, 9 aa, Rattus rattus
AKKMWKKTW

SEQ ID NO: 2 - protein, 9 aa, Rattus rattus
  misc_feature (2)..(2): any amino acid
AXWKEASWK

SEQ ID NO: 3 - protein, 9 aa, Rattus rattus
  misc_feature (2)..(2): any  amino acid
AXRKKKPPD

SEQ ID NO: 4 - protein, 9 aa, Rattus rattus
ADYYWNKNK

SEQ ID NO: 5 - protein, 9 aa, Rattus rattus
  misc_feature (2)..(2): any amino acid
AXKTNKWWK

SEQ ID NO: 6 - protein, 9 aa, Rattus rattus
  misc_feature (2)..(4): any amino acid
AXXXKKYYW

SEQ ID NO: 7 - protein, 9 aa, Rattus rattus
  misc_feature (2)..(3): any amino acid
AXXTKASSK

SEQ ID NO: 8 - protein, 9 aa, Rattus rattus
  misc_feature (2)..(2): any amino acid
AXKFFKISS

SEQ ID NO: 9 - protein, 9 aa, Rattus rattus
AALYSMKVW

SEQ ID NO: 10 - protein, 9 aa, Rattus rattus
AALYYMKIV

SEQ ID NO: 11 - protein, 9 aa, Rattus rattus
  misc_feature (3)..(3): any amino acid
ALXKYAAGG

SEQ ID NO: 12 - protein, 9 aa, Rattus rattus
  misc_feature (3)..(3): any amino acid
ALXKYFAGG

SEQ ID NO: 13 - protein, 9 aa, Rattus rattus
APHKKLLAA

SEQ ID NO: 14 - protein, 9 aa, Rattus rattus
APHKKLLAA

SEQ ID NO: 15 - protein, 9 aa, Rattus rattus
APHKKLVAA

SEQ ID NO: 16 - protein, 9 aa, Rattus rattus
APKIKQPKK

SEQ ID NO: 17 - protein, 9 aa, Rattus rattus
  MISC_FEATURE (3)..(3): any amino acid
APXNKAFFX

SEQ ID NO: 18 - protein, 9 aa, Rattus rattus
  misc_feature (2)..(4): any amino acid
AXXXKNLFF

SEQ ID NO: 19 - protein, 9 aa, Rattus rattus
AGHNDKILM

SEQ ID NO: 20 - protein, 9 aa, Rattus rattus
  misc_feature (4)..(4): any amino acid
AFVXQKVFV

SEQ ID NO: 21 - protein, 9 aa, Rattus rattus
  misc_feature (4)..(4): any amino acid
AFVXQKKFV

SEQ ID NO: 22 - protein, 8 aa, Rattus rattus
  misc_feature (2)..(2): any amino acid
AXHSKVXX

SEQ ID NO: 23 - protein, 8 aa, Rattus rattus
  misc_feature (2)..(2): any amino acid
AXNMQKMM

SEQ ID NO: 24 - protein, 9 aa, Rattus rattus
  misc_feature (2)..(2): any amino acid
AXAMAKMMQ

SEQ ID NO: 25 - protein, 9 aa, Rattus rattus
  misc_feature (2)..(2): any amino acid
AXNMFKMMQ

SEQ ID NO: 26 - protein, 9 aa, Rattus rattus
  misc_feature (3)..(3): any amino acid
ALXHKYPLL

SEQ ID NO: 27 - protein, 9 aa, Rattus rattus
  misc_feature (2)..(3): any amino acid
AXXKAWWLL

SEQ ID NO: 28 - protein, 9 aa, Rattus rattus
  misc_feature (2)..(2): any amino acid
AXIIAKLLX

SEQ ID NO: 29 - protein, 7 aa, Rattus rattus
AKSPKKP

SEQ ID NO: 30 - protein, 9 aa, Rattus rattus
ALYWELAGD

SEQ ID NO: 31 - protein, 7 aa, Rattus rattus
AEWEDDT

SEQ ID NO: 32 - protein, 7 aa, Rattus rattus
AEVFEDD

SEQ ID NO: 33 - protein, 7 aa, Rattus rattus
ADDWDDS

SEQ ID NO: 34 - protein, 7 aa, Rattus rattus
APDWDDE

SEQ ID NO: 35 - protein, 7 aa, Rattus rattus
APWDEDE

SEQ ID NO: 36 - protein, 7 aa, Rattus rattus
AEDEEDW

SEQ ID NO: 37 - protein, 7 aa, Rattus rattus
ADVGDFE

SEQ ID NO: 38 - protein, 7 aa, Rattus rattus
ADVGIFE

SEQ ID NO: 39 - protein, 7 aa, Rattus rattus
AEEDWSD

SEQ ID NO: 40 - protein, 7 aa, Rattus rattus
AYEDDWD

SEQ ID NO: 41 - protein, 7 aa, Rattus rattus
AIDFDDE

SEQ ID NO: 42 - protein, 7 aa, Rattus rattus
ADTWDDD

SEQ ID NO: 43 - protein, 7 aa, Rattus rattus
ADTWDDW

SEQ ID NO: 44 - protein, 7 aa, Rattus rattus
ADNWDDD

SEQ ID NO: 45 - protein, 7 aa, Rattus rattus
AQDEEEW

SEQ ID NO: 46 - protein, 7 aa, Rattus rattus
ATVTEQQ

SEQ ID NO: 47 - protein, 7 aa, Rattus rattus
AIDDDWD

SEQ ID NO: 48 - protein, 7 aa, Rattus rattus
AEDWDDE

SEQ ID NO: 49 - protein, 7 aa, Rattus rattus
ADEEDYW

SEQ ID NO: 50 - protein, 7 aa, Rattus rattus
ADDDEEW

SEQ ID NO: 51 - protein, 7 aa, Rattus rattus
AIDDDIE

SEQ ID NO: 52 - protein, 7 aa, Rattus rattus
ANNWDDD

SEQ ID NO: 53 - protein, 7 aa, Rattus rattus
ADDLADD

SEQ ID NO: 54 - protein, 7 aa, Rattus rattus
AEDDVDD

SEQ ID NO: 55 - protein, 7 aa, Rattus rattus
ANNFDDI

SEQ ID NO: 56 - protein, 7 aa, Rattus rattus
AIDWEEE

SEQ ID NO: 57 - protein, 7 aa, Rattus rattus
AGWDESD

SEQ ID NO: 58 - protein, 7 aa, Rattus rattus
ADDADIV

SEQ ID NO: 59 - protein, 7 aa, Rattus rattus
AVEEDDW

SEQ ID NO: 60 - protein, 7 aa, Rattus rattus
APDEWDD

SEQ ID NO: 61 - protein, 7 aa, Rattus rattus
AWEGDDD

SEQ ID NO: 62 - protein, 7 aa, Rattus rattus
AVDDGDE

SEQ ID NO: 63 - protein, 7 aa, Rattus rattus
AGDEDWP

SEQ ID NO: 64 - protein, 7 aa, Rattus rattus
AANFDEN

SEQ ID NO: 65 - protein, 7 aa, Rattus rattus
AGEDEWE

SEQ ID NO: 66 - protein, 7 aa, Rattus rattus
AAEDDEW

SEQ ID NO: 67 - protein, 7 aa, Rattus rattus
ADWPNED

SEQ ID NO: 68 - protein, 7 aa, Rattus rattus
ADEYDWP

SEQ ID NO: 69 - protein, 7 aa, Rattus rattus
AEGPDDL

SEQ ID NO: 70 - protein, 7 aa, Rattus rattus
APWTDDD

SEQ ID NO: 71 - protein, 7 aa, Rattus rattus
AETFDED

SEQ ID NO: 72 - protein, 7 aa, Rattus rattus
AFDDDFP

SEQ ID NO: 73 - protein, 7 aa, Rattus rattus
ANYDDDW

SEQ ID NO: 74 - protein, 7 aa, Rattus rattus
ANYDDDW

SEQ ID NO: 75 - protein, 7 aa, Rattus rattus
AEIDIWE

SEQ ID NO: 76 - protein, 7 aa, Rattus rattus
APEEEEW

SEQ ID NO: 77 - protein, 7 aa, Rattus rattus
AEDDDFT

SEQ ID NO: 78 - protein, 7 aa, Rattus rattus
AQWDDED

SEQ ID NO: 79 - protein, 7 aa, Rattus rattus
AENEDYD

SEQ ID NO: 80 - protein, 7 aa, Rattus rattus
ADEFEDD

SEQ ID NO: 81 - protein, 7 aa, Rattus rattus
ADIDFYF

SEQ ID NO: 82 - protein, 7 aa, Rattus rattus
ADDSWED

SEQ ID NO: 83 - protein, 7 aa, Rattus rattus
AWDEFEV

(19) United States
(12) Patent Application Publication
(10) Pub. No.: US 2005/0222041 A1
(43) Pub. Date: Oct. 6, 2005

(54) Ncam binding compounds

(75) Inventors: Lars Christian Ronn Biilmann, Copenhagen (DK); Marianne Olsen, Roskilde (DK); Elisabeth Bock, Charlottenlund (DK); Vladimir Berezin, Copenhagen (DE); Flemming Martin Poulsen, Frederiksberg (DK); Arne Valdemar Holm, Skodsborg (DK); Vladislav Soroka, Hellerup (DK); Soren Ostergaard, Bronshoj (DK)

Correspondence Address: BROWDY AND NEIMARK, P.L.L.C.;624 NINTH STREET, NW, SUITE 300, WASHINGTON, DC, 20001-5303, US

(73) Assignee: ENKAM Pharmaceuticals A/S, Coperhagen (DK)

(21) Appl. No.: 10/488,517

(22) PCT Filed: Sep. 4, 2002
(86) PCT No.: PCT/DK02/00574

§ 371 Date: Dec. 1, 2004

(30) Foreign Application Priority Data
Sep. 5, 2001 (DK) PA 2001 01299

(51) Int. Cl. A61K 38/08; C07K 7/06

(52) U.S. Cl. 514/16; 530/329

(57) ABSTRACT

The present invention concerns an NCAM (neural cell adhesion molecule) binding peptide capable of stimulating NCAM signalling and/or interfering with cell adhesion. The compound may be used for the treatment of diseases or conditions of the central and peripheral nervous system.

### TECHNICAL BACKGROUND OF THE INVENTION

The neural cell adhesion molecule (NCAM) is believed to play an important role in formation of neuronal connections in the developing nervous system. In addition considerable evidence suggests that in the adult nervous system, NCAM is involved in learning and regeneration.

NCAM is expressed as three major isoforms in the nervous system of which two, NCAM-180 (NCAM-A) and NCAM-140 (NCAM-B) are transmembrane, while the third, NCAM-120 (NCAM-C) is linked to the membrane via a GPI-anchor. In addition soluble forms of NCAM may be generated by truncation and shedding.

NCAM mediates cell-cell adhesion through a homophilic (NCAM-NCAM) mechanism. In addition, NCAM binds heterophilically to other cell surface receptors and extracellular matrix components, including heparan sulphate proteoglycans (Cole and Glaser, 1986) and the cell adhesion molecules L1 and TAG-1/axonin-1 (Holley and Yu, 1987)(Milev et al., 1996). The extracellular part of NCAM is composed of five immunoglobulin-like homology modules (Ig) and two fibronectin type III like modules (FN3). The modules mediating homophilic NCAM-binding have not been unequivocally identified. Reciprocal interactions, either between the IgIII modules (Rao et al., 1994) or between all five Ig-modules of two NCAM molecules (Ranheim et al., 1996), have been suggested to be responsible for homophilic NCAM-binding. Recently, binding between the recombinant NCAM modules IgI and IgI has been demonstrated by plasmon surface resonance analysis (Kiselyov et al., 1997) and structurally characterised by means of nuclear magnetic resonance (NMR) (Thomsen et al., 1996; Jensen et al., 1999) and X-ray crystallography (Kasper et al., 2000) suggesting that homophilic NCAM binding is mediated by a double reciprocal interaction between the IgI- and IgII-modules of two NCAM-molecules.

Homophilic NCAM binding has been shown to initiate a signalling cascade (Kolkova et al., 2000) involving, activation of a fibroblast growth factor receptor (FGF-R)-dependent pathway (Doherty and Walsh, 1996) and the Ras-MAP-kinase pathway (Schmid et al., 1999). NCAM has been hypothesised to bind the FGF-R by interacting with the so-called CAM homology domain (CHD), a sequence in the FGF-R with homology to NCAM and the cell adhesion molecules L1 and N-cadherin. NCAM mediated induction of neurite outgrowth has been shown to be dependent on voltage dependent calcium channels (VDCCs). Thus, NCAM-dependent neurite outgrowth can be inhibited by VDDC antagonists (Doherty et al., 1991) and NCAM antibodies in high concentrations has been shown to induce an increase in intracellular calcium by spectrofluorometry (Schuch et al., 1989).

The present invention concerns compounds capable of modulating NCAM functions, such as cell aggregation, neurite outgrowth, and regulation of intracellular calcium.

### SUMMARY

Accordingly, the present invention concerns compounds which are capable of modulating proliferation, induce differentiation, and promote regeneration, neuronal plasticity and survival of cells expressing NCAM. These changes are believed to be caused via a stimulation of NCAM signalling.

In one aspect the present invention concerns an NCAM (neural cell adhesion molecule) binding compound capable of stimulating NCAM signalling and/or interfering with cell adhesion, comprising a peptide having a sequence of the formula L1-A-L2-B-L3-C-L4, wherein
A, B or C are any amino acid residue, with the proviso that at least B or C is an acidic amino acid residue, and at least A, B, or C is a hydrophobic amino acid residue or Y, or
A, B or C are any amino acid residue, with the proviso that at least B or C is a basic amino acid residue, and at least A, B, or C is a hydrophobic amino acid residue or Y, and
L1, L2, L3 and L4 are individually selected from a chemical bond or an amino acid sequence having n amino acid residues, wherein n is an integer of from 0 to 5.

In the present context the standard one-letter code for amino acid residues as well as the standard three-letter code for amino acids are applied.

In another embodiment the invention discloses a compound comprising at least seven amino acid residues,
of the formula A-P2-P3-P4-P5-P6-P7, wherein
A is the amino acid residue A
P2 is one amino acid selected from the group consisting of the amino acid residues D, E, P, I, Y, V, G, F, Q, A, N, W and T;
P3 is one amino acid selected from the group consisting of the amino acid residues D, E, I, W, V, N, T, G, an hydrophilic amino acid and Y;
P4 is one amino acid selected from the group consisting of the amino acid residues D, E, W, F, T, L, A, G, P, S, Y and T,
P5 is one amino acid selected from the group consisting of the amino acid residues D, E, W, A, V, G, E, N, I and F,
P6 is selected from the group consisting of the amino acid residues D, E, F, S, W, Q, Y and I,
P7 is selected from the group consisting of the amino acid residues D, E, N, T, S, W, Q, I, V, P, L and F.

In another embodiment the invention relates to a compound comprising at least nine amino acid residues,
of the formula A-P2-P3-P4-P5-P6-P7-P8-P9, wherein
A is the amino acid residue A
P2 is one amino acid selected from the group consisting of the amino acid residues K, D, A, L, P, G, and F, or a bond,
P3 is one amino acid selected from the group consisting of the amino acid residues K, W, R, Y, L, N, H, and V, and S, or a bond,
P4 is one amino acid selected from the group consisting of the amino acid residues K, M, Y, T, F, I, N, S, and H and P, or a bond,
P5 is one amino acid selected from the group consisting of the amino acid residues W, E, K, N, F, S, Y, V, D, Q, and A, or a bond,
P6 is one amino acid selected from the group consisting of the amino acid residues K, A, N, M, F, O, L, V, Y, and W, or a bond,
P7 is one amino acid selected from the group consisting of the amino acid residues K, S, P, W, Y, I, A, L, V, F, and M, or a bond,
P8 is one amino acid selected from the group consisting of the amino acid residues K, T, W, P, N, Y, S, V, I, G, A, F, L, and M, or a bond,
P9 is selected from the group consisting of the amino acid residues W, K, D, S, V, G, A, F, M, Q, and L, or a bond.

It is preferred that at most two of the positions are occupied by a bond.

Further the invention concerns the use of a compound as mentioned above and a pharmaceutical composition, comprising one or more of said compounds and the use of said pharmaceutical composition as well as a method for treatment using the compound. The invention also relates to a prosthetic nerve guide, characterised in that said nerve guide comprises one or more of the compounds according to the invention.

### FIGURES

FIG. 1. shows the effect of an NCAM Binding Peptide (NBP10) on cell aggregation. A) Number of aggregates in the presence of NBP10 as monomer (m) (diamonds), dendrimer (d) (squares) or BSA-bound 20-mer (circles) in the indicated concentrations. Data points are means+/−SEM of 3-7 independent experiments. B-F) Micrographs of hippocampal aggregate cultures grown for 24 hours in the absence (B) or presence of NBP10m 60 μM (C), NBP10d 1 μM (D), NBP10m 200 μM (E) or NBP10d 6 μM (F). Bar: 100 μm. G-J) Micrographs at high magnification of cultures grown in the absence (G) or presence of NBP10d 2 μM (H), NBP10m 60 μM (I) or NBP10m 200 μM (J). Bar: 25 μm.

FIG. 2. shows the Effect of NBP10 on neurite outgrowth induced by homophilic NCAM binding. A) Length of neurites from primary hippocampal neurons grown on monolayers of NCAM-transfected fibroblasts (diamonds) or monolayers without NCAM expression (circles) in the presence of NBP10-BSA in the indicated concentrations. Data are normalised to control cultures of neurons grown on fibroblast monolayers without NCAM expression. Each data point represents means of 2-5 independent experiments. B) Length of neurites from hippocampal cells grown on monolayers without NCAM expression (LVN, white columns) or monolayers of NCAM-transfected fibroblasts (LBN, black columns) in the presence of NBP10-BSA; NBP9-BSA or BSA alone (100 μg/ml). Data points represent means of 3-5 independent experiments. *P<0.05 when compared to LVN control. ++P<0.01 when compared to LBN control, Students T-test.

FIG. 3 Effect of NBP10 on neurite outgrowth. A,B) Micrographs of primary hippocampal neurons grown for 24 hours in the absence (A) or presence (B) of NBP10d (1 μM). Bar: 20 μm. C) Length of neurites from primary hippocampal neurons grown in the presence of NBP10m (circles) or NBP10d (diamonds) in the indicated concentration. Data points are means+/−SEM of 3-4 independent experiments. D) Length of neurites in the presence of NBP10-BSA, NBP9-BSA or BSA alone (20 μM). Data points are means+/−SEM of 3-4 independent experiments. *P<0.05, paired t-test. E) Effect on neurite outgrowth of monomeric NBP10-peptide modified by single substitutions with alanine or phenylalanine. *p<0.05 when compared to the effect of NBP10, Students T-test. F) Effect of putative inhibitors of NCAM dependent signal transduction on neurite outgrowth induced by NBP10d (1 μM). aFGFR: rabbit antiserum (1:1000) raised against a synthetic peptide corresponding to amino acids 119-144 of the chicken FGF-receptor situated close to the so-called CAM homology domain, Nif: nifidipine (10 μM), MVIIA: (&conotoxin MVIIA (1 μM). P38inh: SB203580 (1 μM), Mekinh: PD98059 (10 μM). *P<0.05, **P<0.01, ***P<0.001 when compared to effect of NBP10d, Paired T-test.

FIG. 4: shows the effect of NBP10 on intracellular calcium in PC12E2 cells. A-D) Micrographs illustrating the effect of NBP10d application (50 μM) on the intracellular calcium concentration in fura-2-AM loaded PC12E2 cells. Under these conditions, an increase in intracellular calcium will be reflected by an increased fluorescence at an excitation wavelength, of 340 nm and a concomitant decrease in fluorescence at an excitation wavelength of 380 nm. A) Fluorescence image obtained by excitation at 340 nm before application of NBP10d. Bar: 25 μm. B) Fluorescence image obtained by excitation at 340 nm 50 sec after application of NBP10d. C) Fluorescence image obtained by excitation at 380 nm before application of NBP10d. D) Fluorescence image obtained by excitation at 380 nm 50 sec after application of NBP10d. E) Time course of changes in the concentration of intracellular calcium in PC12E2 cells after application of NBP10d. The intracellular concentration of calcium was calculated from the ratio between fluorescence images obtained by excitation at 340 nm (A,B) and 380 nm (C,D). Each trace represents one individual cell. Representative of 6 independent experiments.

FIG. 5: Mapping of the binding site of NBP10d onto the structure of NCAM-IgI. Amino acid residues of IgI exhibiting chemical shift changes >0.01 p.p.m. for ¹H, and >0.07 p.p.m. for ¹⁵N shifts upon binding to NBP10d are shown.

### DETAILED DESCRIPTION OF THE INVENTION

The compound according to the invention is suitably used for the promotion of cell differentiation and modulation of proliferation of neural cells and neuronal plasticity, and stimulation of survival and regeneration of neuronal cells.

Substances with the potential to promote neurite outgrowth as well as stimulate survival, regeneration and modulate proliferation of neuronal cells, such as certain endogenous trophic factors, are prime targets in the search for compounds that facilitate for example neuronal regeneration and other forms of neuronal plasticity. To evaluate the potential of the present compound, the ability to stimulate NCAM signalling, interfere with cell adhedion, stimulate neurite outgrowth, proliferation and regeneration, the survival of neuronal cells may be investigated. Compounds of the present invention capable of binding to one or more positions of the NCAM molecule, in particular positions in NCAM Ig1 to NCAM Ig5, are shown to promote neurite outgrowth and to effect neuronal proliferation and are therefore considered to be good promoters of regeneration of neuronal connections, and thereby of functional recovery after damages as well as promoters of neuronal function in other conditions where such an effect is required.

In the present context “differentiation” is related to the processes of maturation of neurons and extension of neurites which takes place after the last cell division of said neurons has ended. The compounds of the present invention may be capable of stopping cell division and initiate maturation and/or extension.

In the present invention a compound is considered promising when it is capable of doubling the neurite outgrowth of cells cultured under conditions as described in Example 3 when compared to control cells, such as improving neurite outgrowth three-fold, such as four-fold, for example five fold, such as six-fold.

Further, in the present context the wording “stimulate/promoting survival” is used synonymously with the wording “preventing cell death” or “neuroprotection”. By stimulating/promoting survival it is possible to prevent diseases or prevent further degeneration of the nervous system in individuals suffering from a degenerative disorder.

“Survival” refers to the process, wherein a cell has been traumatised and would under normal circumstances, with a high probability die, if not the compound of the invention was used to prevent said cell from degenerating, and thus promoting or stimulating survival of said traumatised cell.

By “stimulating NCAM signalling” is meant a molecule capable of initiating the production and/or activation of a cascade of messenger molecules leading to a physiological response of the cell, such as an increase in neurite length.

The invention further provides for a compound capable of “interfering with cell adhesion”. This refers to the process wherein cells are attracted to one another and where the present compound is capable of either stimulating or inhibit said attraction.

By the term “modulation” is meant a change, such as either a stimulation or an inhibition.

The term “ligand” is defined as a compound which binds and mimics the compound of the present invention. The ligand may also inhibit naturally occurring interactions, such as by binding to parts of NCAM which are not a part of the binding sites, and wherein the interference is merely a steric interference.

The compound according to the invention also relates to the prevention of neuronal cell death. Peripheral nerve cells possess to a limited extent a potential to regenerate and reestablish functional connections with their targets after various injuries. However, functional recovery is rarely complete and peripheral nerve cell damage remains a considerable problem. In the central nervous system, the potential for regeneration is even more limited. Therefore, the identification of substances with the ability to prevent neuronal cell death in the peripheral and the central nervous system is significant and of great commercial value.

Novel Compounds

The invention thus relates to novel compounds, i.e. a compound comprising an NCAM (neural cell adhesion molecule) binding compound capable of stimulating NCAM signalling and/or interfering with cell adhesion, comprising a peptide having a sequence of the formula L1-A-12-B-L3-C-L4, wherein
A, B or C are any amino acid residue, with the proviso that at least B or C is an acidic amino acid residue, and at least A, B, or C is a hydrophobic amino acid residue or Y, or
A, B or C are any amino acid residue, with the proviso that at least B or C is a basic amino acid residue, and at least A, B, or C is a hydrophobic amino acid residue or Y, and
L1, L2, L3 and L4 are individually selected from a chemical bond or an amino acid sequence having n amino acid residues, wherein n is an integer of from 0 to 5.

In one embodiment at least one of B, C, or L denotes a hydrophobic amino acid residue and in another embodiment the hydrophobic group is an aromatic amino acid residue.

In one aspect the invention concerns a compound comprising a peptide having at least seven amino acid residues,
of the formula A-P2-P3-P4-P5-P6-P7, wherein
A is the amino acid residue A
P2 is one amino acid selected from the group consisting of the amino acid residues D, E, P, I, Y, V, G, F, Q, A, N, W and T;
P3 is one amino acid selected from the group consisting of the amino acid residues D, E, I, X, W, V, N, T, G, an hydrophilic amino acid and Y;
P4 is one amino acid selected from the group consisting of the amino acid residues D, E, W, F, X, T, L, A, G, P, S, Y and T,
P5 is one amino acid selected from the group consisting of the amino acid residues D, E, W, A, V, G, N, I and F,
P6 is selected from the group consisting of the amino acid residues D, E, F, S, W, Q, Y and I,
P7 is selected from the group consisting of the amino acid residues D, E, N, T, S, W, Q, I, V, P, L and F.

In one preferred embodiment P2 is selected from the group consisting of D, E, P, I, Y, V, G, Q, F, W and T or from the group consisting of D, E, P, I, Y, V, N, G, F, W and T or from the group consisting of D, E, P, I, G, N, Q, W, A, V and F or from the group consisting of D, E, P, I, Y, V, N, F, Q, W and T or from the group consisting of D, E, P, I, Y, V, N, F, G, Q and W.

An even more preferred embodiment is when P2 is selected from the group consisting of D, E, P, I, G, N, O, A and V.

In a preferred embodiment P3 is selected from the group consisting of D, E, I, W, V and N or from the group consisting of D, E, I, W, V and T.

In another embodiment P3 is selected from the group consisting of D, E, W or N or from the group consisting of D, E, V or I.

In a preferred embodiment P4 is selected from the group consisting of D, E, W, F, A, G, P, Y and T or from the group consisting of D, E, W, F, P, T, Y, S and L or from the group consisting of D, E, W, F, G, T, Y, S and L or from the group consisting of D, E, W, L, A, G, P, Y and T or from the group consisting of D, E, W, F, L, A, G, P and Y.

In another preferred embodiment P4 is selected from the group consisting of the amino acid residues D, E, W, F and P. In another preferred embodiment P4 is selected from the group consisting of the amino acid residues D, E and W or from the group consisting of D, E and T.

In one embodiment P5 is selected from the group consisting of the amino acid residues D, E, W, N, I, F, A, V and G or from the group consisting of the amino acid residues D, E, W, I, A, V, N, and G.

In a further embodiment P5 is selected from the group consisting of the amino acid residues. D, E, W, V, I and F.

In one embodiment P6 is selected from the group consisting of the amino acid residues D, E, W, Y, F, I, and S or from the group consisting of the amino acid residues D, E, W, F, Y, I, and Q.

In a further embodiment P6 is selected from the group consisting of the amino acid residues D, E, F and Y.

In yet a further embodiment P6 is selected from the group consisting of the amino acid residues D, E, Q and W.

In one embodiment of the invention. P7 is selected from the group consisting of the amino acid residues D, E, W, P, V, T, I, F, S, N, and Q or from the group consisting of D, E, T, S, W, I, V, P, L and F or from the group consisting of D, E, T, Q, W, I, V, P, L and F or from the group consisting of D, E and at least one of S, W, Q, I, V, P and F.

A preferred embodiment of the present invention, is wherein
P2 is selected from the group consisting of the amino acid residues D, E, P, I, G, N, Q, A and V,
P3 is selected from the group consisting of the amino acid residues D, E, I, W, V, N and T,
P4 is selected from the group consisting of the amino acid residues D, E, T and W,
P5 is selected from the group consisting of the amino acid residues D, E, W, V, I and F,
P6 is selected from the group consisting of the amino acid residues D, E, Q and W, and
P7 is selected from the group consisting of the amino acid residues S, W, O, I, V, P and F.

Another aspect of the invention concerns a compound comprising a peptide,
of the formula A-P2-P3-P4-P5-P6-P7-P8-P9, wherein
A is the amino acid residue A
P2 is selected from the group consisting of the amino acid residues K, D, A, L, P, G, and F, or a bond,
P3 is selected from the group consisting of the amino acid residues K, W, R, Y, L, N, H, S, I, and V, or a bond,
P4 is selected from the group consisting of the amino acid residues K, M, Y, T, F, I, N, S, P, and H, or a bond,
P5 is selected from the group consisting of the amino acid residues W. E, K, N, F, S, Y, V, D, Q, and A or a bond,
P6 is selected from the group consisting of the amino acid residues K, A, N, M, F, Q, L, V, Y, and W, or a bond,
P7 is selected from the group consisting of the amino acid residues K, S, P, W, Y, I, A, L, V, F, and M, or a bond,
P8 is selected from the group consisting of the amino acid residues K, T, W, P, N, Y, S, V, I, G, A, F, L, and M, or a bond,
P9 is selected from the group consisting of the amino acid residues. W, K, D, S, V, G, A, F, M, O, and L or a bond.

It is preferred that at most two positions are a bond, wherein bond means chemical linkage or chemical bond. Preferably P8 and/or P9 is a bond.

In one embodiment each of the positions P2-P9 are selected individually from the group consisting of the amino acid residues T, N and Y.

In a preferred embodiment P2 is selected from the group consisting of the amino acid residues K, L, P, A, F, D, and G, or from the group consisting of the amino acid residues P, L, K, and A.

In another embodiment P2 is selected from the group consisting of the amino acid residues D and K (secondary charged amino acids).

In yet another embodiment P3 is selected from the group consisting of the amino acid residues K, H, V, L, and I or from the group consisting of the amino acid residues K, W, R, Y, L, H and V or from the group consisting of the amino acid residues W, L or Y.

In a further embodiment P3 is selected from the group consisting of the amino acid residues K, H and Y.

The invention further relates to a compound, wherein P4 is selected from the group consisting of the amino acid residues K, T, I, M, Y, N, P, and F or from the group consisting of the amino acid residues K, M, Y, T, F, I, S and H or from the group consisting of the amino acid residues K, M, Y, T, F, I, N and H or from the group consisting of the amino acid residues K, T, I, M, Y, N, S, and H.

Furthermore, the invention concerns a compound, wherein P4 is selected from the group consisting of the amino acid residues K, T and I or from the group consisting of the amino acid residues M, F and I.

In another embodiment P5 is selected from the group consisting of the amino acid residues K, A, F, W, Q, V, D, E, and S, or from the group consisting of the amino acid residues W, E, K, N, F, S, Y, V, D and A or from the group consisting of the amino acid residues W, K, N, F, S, Y, V, D, Q and A or from the group consisting of the amino acid residues W, E, K, N, F, S, Y. V, Q and A or from the group consisting of the amino acid residues K, A, F, W, Q, V, D, S, and N.

Furthermore, in one embodiment of the invention P5 is selected from the group consisting of the amino acid residues W, F, V or Y.

In yet another embodiment P5 is selected from the group consisting of the amino acid residues K, A, F, W, V, S, Y, and D.

In another embodiment P6 is selected from the group consisting of the amino acid residues K, A, M, F, Q, L, V, Y and W or from the group consisting of the amino acid residues K, A, N, M, F, L, V, Y and W.

In a further embodiment P6 is selected from the group consisting of the amino acid residues A, M, F, L, V and W.

A preferred embodiment of P6 is selected from the group consisting of the amino acid residues K, A, or N and Q or from the group consisting of the amino acid residues K, A, N, and M.

In one embodiment P7 is selected from the group consisting of the amino acid residues K, P, L, V, I, W, S, A, F, and Y or from the group consisting of the amino acid residues K, P, W, Y, I, A, L, V, F and M or from the group consisting of the amino acid residues P, L, V, F, W, S, F, A, and M, or Y or from the group consisting of the amino acid residues P, W, I, A, L, V, F and M, or Y.

In one embodiment P8 is selected from the group consisting of the amino acid residues T, W, P, N, Y, S, V, I, G, A, F, L and M or from the group consisting of the amino acid residues W, P, V, I, A, F, L and M.

In another embodiment P8 is selected from the group consisting of the amino acid residues W, P, V, I, A, F and L or Y or from the group consisting of the amino acid residues at least one of T, N and Y.

In one embodiment P8 is Y.

In one embodiment of the invention P9 is selected from the group consisting of the amino acid residues W, K, D, S, V, G, A, F, M, Q and L or from the group consisting of the amino acid residues W, V, A, F, M and L.

In a further embodiment P9 is selected from the group consisting of the amino acid residues N, Y and S or from the group consisting of the amino acid residues K, W, D, and S.

It is preferred that at least one of the positions P2-P9 is occupied by a hydrophobic amino acid residue. Furthermore, it is preferred that at least one of the positions P2-P9 is occupied by a hydrophilic amino acid residue. Also, it is preferred that at least one of the positions P2-P9 is occupied by an acidic or a basic amino acid residue.

In a preferred embodiment the compound comprises a peptide comprising one or more of the following amino acid sequences:
SEQ ID NO:1 or SEQ ID NO:2 or SEQ ID NO:3 or SEQ ID NO:4 or SEQ ID NO:5 or SEQ ID NO:6 or SEQ ID NO:7 or SEQ ID NO:8, or SEQ ID NO:9, or SEQ ID NO:10, or SEQ ID NO:11, or SEQ ID NO:12, or SEQ ID NO:13, or SEQ ID NO:14, or SEQ ID NO:15, or SEQ ID NO:16, or SEQ ID NO:17, or SEQ ID NO:18, or SEQ ID NO:19, or SEQ ID NO:20, or SEQ ID NO:21, or SEQ ID NO:22, or SEQ ID NO:23, or SEQ ID NO:24, or SEQ ID NO:25, or SEQ ID NO:26, or SEQ ID NO:27, or SEQ ID NO:28, or SEQ ID NO:29, or SEQ ID NO:30, or SEQ ID NO:31, or SEQ ID NO:32, or SEQ ID NO:33, or SEQ ID NO:34, or SEQ ID NO:35, or SEQ ID NO:36, or SEQ ID NO:37, or SEQ ID NO:38, or SEQ ID NO:39′, or SEQ ID NO:40, or SEQ ID NO:41, or SEQ ID NO:42, or SEQ ID NO:43, or SEQ ID NO:44, or SEQ ID NO:45, or SEQ ID NO:46, or SEQ ID NO:47, or SEQ ID NO:48, or SEQ ID NO:49, or SEQ ID NO:50, or SEQ ID NO:51, or SEQ ID NO:52, or SEQ ID NO:53, or SEQ ID NO:54, or SEQ ID NO:55, or SEQ ID NO:56, or SEQ ID NO:57, or SEQ ID NO:58, or SEQ ID NO:59, or SEQ ID NO:60, or SEQ ID NO:61, or SEQ ID NO:62, or SEQ ID NO:63, or SEQ ID NO:64, or SEQ ID NO:65, or SEQ ID NO:66, or SEQ ID NO:67, or SEQ ID NO:68, or SEQ ID NO:69, or SEQ ID NO:70, or SEQ ID NO:71, or SEQ ID NO:72, or SEQ ID NO:73, or SEQ ID NO:74, or SEQ ID NO:75, or SEQ ID NO:76, or SEQ ID NO:77, or SEQ ID NO:78, or SEQ ID NO:79, or SEQ ID NO:80, or SEQ ID NO:81, or SEQ ID NO:82, or SEQ ID NO:83 or more preferred SEQ ID NO:31, SEQ ID NO:32, or SEQ ID NO:33, or SEQ ID. NO:34, or SEQ ID NO:35, or SEQ ID NO:36, or SEQ ID NO:37, or SEQ ID NO:38, or SEQ ID NO:39, or SEQ ID NO:40, or SEQ ID NO:41, or SEQ ID NO:42, or SEQ ID NO:43, or SEQ ID NO:44, or SEQ ID NO:45, or SEQ ID NO:46, or SEQ ID NO:47, or SEQ ID NO:48, or SEQ ID NO:49, or SEQ ID NO:50, or SEQ ID NO:51, or SEQ ID NO:52, or SEQ ID NO:53, or SEQ ID NO:54, or SEQ ID NO:55, or SEQ ID NO:56, or SEQ ID NO:57, or SEQ ID NO:58, or SEQ ID NO:59, or SEQ ID NO:60, or SEQ ID NO:61, or SEQ ID NO:62, or SEQ ID NO:63, or SEQ ID NO:64, or SEQ ID NO:65, or SEQ ID NO:66, or SEQ ID NO:67, or SEQ ID NO:68, or SEQ ID NO:69, or SEQ ID NO:70, or SEQ ID NO:71, or SEQ ID NO:72, or SEQ ID NO:73, such as SEQ ID NO:31, SEQ ID NO:32, or SEQ ID NO:33, or SEQ ID NO:34, or SEQ ID NO:35, or SEQ ID NO:36, or SEQ ID NO:37, or SEQ ID NO:38, or SEQ ID NO:39, or SEQ ID NO:40, or SEQ ID NO:41, or SEQ ID NO:42, or SEQ ID NO:43, or SEQ ID NO:44, or SEQ ID NO:45, or SEQ ID NO:46, or SEQ ID NO:47, or SEQ ID NO:48, or SEQ ID NO:49, or SEQ ID NO:50, or SEQ ID NO:51, or SEQ ID NO:52, or SEQ ID NO:53, or SEQ ID NO:54, or SEQ ID NO:55, or SEQ ID NO:56, or SEQ ID NO:57, or SEQ ID NO:58.

In a further embodiment the compound may comprise other chemical entities, such as sugar, cholesterol, and fatty acid. Preferably, the chemical entity is bound to the N-terminal or C-terminal of the peptide of the compound.

It is an aspect of the present invention that the compounds are capable of binding to the NCAM molecule at either a homophilic or a heterophilic binding site.

Without being bound by theory, the present inventors believe that active ligands to the NCAM Ig1 and/or the NCAM Ig2 and/or NCAM Ig3 and/or NCAM Ig4 and/or NCAM Ig5 domain are ligands which bind to the NCAM Ig1 domain and/or the NCAM Ig2 and/or NCAM Ig3 and/or NCAM 194 and/or NCAM Ig5 domain and thus trigger a conformational change of the domain resulting in a signalling cascade being initiated, wherein said signalling results in a physiological change in the cell, such as influencing proliferation of cells and/or neurite outgrowth. Thus, a compound according to the invention may be any compound of the compounds described above which can trigger a conformational change of the NCAM Ig1 domain and/or the NCAM Ig2 and/or NCAM Ig3 and/or NCAM Ig4 and/or NCAM Ig5 domain resulting in a downstream signalling.

Thus, in one embodiment, the present compound is capable of binding to the NCAM Ig1 domain, more specifically binding to the homophilic binding site of the Ig1-Ig2 domains which is constituted by the Ig1 domain. Further, the compound of the invention may be a peptide capable of binding to the NCAM Ig2 binding site on the NCAM Ig1 domain. In one aspect of the invention the compound is capable of binding to a binding site on the NCAM Ig1 domain, wherein the binding site is different from the NCAM Ig2 binding site.

In another embodiment the present compound is capable of binding to the NCAM Ig2 domain, more specifically binding to the homophilic binding site of the Ig1-Ig2 domains, which is constituted by the Ig2 domain.

According to the invention the present compound may be a peptide which binds to the NCAM Ig1 domain through a binding motif which comprises at least 2 basic amino acid residues. Peptides comprising at least 2 basic amino acid residues within a sequence of 10 amino acid residues, preferably within a sequence of 3 amino acid residues are within the scope of the present invention.

It is also within the scope of the invention to provide a compound capable of binding to the NCAM Ig3 domain. In one such embodiment the compound is capable of binding to the homophilic binding site of the Ig3 domain.

In another embodiment of the invention the compound is capable of binding to the NCAM Ig4 domain, such as capable of binding to a homophilic or heterophilic binding site of the Ig4 domain, such as the homopihilic binding site capable of binding Ig2 domain, or the heterophilic binding site which binds to L1 cell adhesion molecule.

In a further embodiment the present compound is capable of binding to the NCAM Ig5 domain, such as capable of binding to the homophilic or heterophilic binding site of the Ig5 domain, such as the homopihilic binding site capable of binding Ig1 domain, or the heterophilic binding site being any heterophilic partner, such as a receptor for polysialic acid linked to the Ig5 domain.

It is within the scope of invention that the compound is capable of binding to the NCAM FN3,1 domain either through a homophilic or a heterophilic binding site. Thus, in one embodiment the compound is capable of binding to a heterophilic binding site of the NCAM FN3,1 domain. In a further aspect of the invention the compound is capable of binding to the NCAM FN3,2 domain, such as a compound being capable of binding to a heterophilic binding site of the NCAM FN3,2 domain. In yet a further embodiment the one of the binding sites is a heterophilic binding site, such as a receptor for ATP.

Throughout the description and claims either the three letter code or the one letter code for natural amino acids are used. Where the L or D form has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms.

Where nothing is specified it is to be understood that the C-terminal amino acid of a polypeptide of the invention exists as the free carboxylic acid, this may also be specified as “—OH”. However, the C-terminal amino acid of a compound of the invention may be the amidated derivative, which is indicated as “—NH₂”. Where nothing else is stated the N-terminal amino acid of a polypeptide comprise a free amino-group, this may also be specified as “H-”.

Where nothing else is specified amino acid can be selected from any amino acid, whether naturally occurring or not, such as alfa amino acids, beta amino acids, and/or gamma amino acids. Accordingly, the group comprises but are not limited to: Ala, Val, Leu, Ile, Pro, Phe, Trp, Met, Gly, Ser, Thr, Cys, Tyr, Asn, Gln, Asp, Glu, Lys, Arg, His Aib, Nal, Sar, Orn, Lysine analogues, DAP, DAPA and 4Hyp.

Also, according to the invention modifications of the compounds/peptides may be performed, such as glycosylation and/or acetylation of the amino acids.

The compound of the invention may have a length which varies, thus accordingly the compound may have a length of between 3-100 amino acid residues, such as 3-50 amino acid residues, for example 3-30 amino acid residues, such as 3-20 amino acid residues.

In another embodiment the present compound may have a length of between 4-100 amino acid residues, such as 4-50 amino acid residues, for example 4-30 amino acid residues, such as 4-20 amino, acid residues.

In a further embodiment the compound is having a length of between 5-100 amino acid residues, such as 5-50 amino acid residues, for example 5-30 amino acid residues, such as 5-20 amino acid residues.

In yet a further embodiment the compound is having a length of between 6-100 amino acid residues, such as 6-50 amino acid residues, for example 6-30 amino acid residues, such as 6-20 amino acid residues.

The invention also discloses a compound having a length of between 7-100 amino acid residues, such as 7-50 amino acid residues, for example 7-30 amino acid residues, such as 7-20 amino acid residues.

The compound of the invention may additionally have a length of between 8-100 amino acid residues, such as 8-50 amino acid residues, for example 8-30 amino acid residues, such as 8-20 amino acid residues.

The compound having a length of between 9-100 amino acid residues, such as 9-50 amino acid residues, for example 9-30 amino acid residues, such as 9-20 amino acid residues is also within the scope of the invention.

In another embodiment the compound is having a length of between 10-100 amino acid residues, such as 10-50 amino acid residues, for example 10-30 amino acid residues, such as 10-20 amino acid residues.

The compound used according to the invention is preferably an oligomer (multimer) of monomers, wherein each monomer is as defined for the compound above. Particularly, multimeric peptides, such as dendrimers may form conformational determinants or clusters due to the presence of multiple flexible peptide monomers. In one embodiment the compound is a dimer. In a more preferred embodiment the compound is a dendrimer, such as four peptides linked to a lysine backbone, or coupled to a polymer carrier, for example a protein carrier, such as BSA. Polymerisation such as repetitive sequences or attachment to various carriers are well-known in the art, e.g. lysine backbones, such as lysine dendrimers carrying 4 peptides, 8 peptides, 16 peptides, or 32 peptides. Other carriers may be lipophilic dendrimers, or micelle-like carriers formed by lipophilic derivatives, or starburst (star-like) carbon chain polymer conjugates.

The compound preferably comprises monomers independently capable of stimulating NCAM receptor signalling and/or interfering with cell adhesion of cells presenting NCAM, and/or NCAM ligand/counter-receptor presenting cells.

The individual monomers may be homologous, i.e. identical to one another, or the individual monomers may be heterologous, i.e. different from one another. The latter type of monomers may comprise at least two different monomers. In general dimers and multimers may comprise two or more identical monomers, or two or more monomers different from one another.

The compound according to the invention preferably has a binding affinity (Kd value/equilibrium constant) to NCAM in the range of between 10⁻² M to 10⁻¹⁰ M, 10⁻³ M to 10⁻⁵ M, such as preferably in the range of between 10⁻⁴ to 10⁻⁵M. According to the present invention the binding affinity is determined by one of the following assays of either surface plasmon resonance analysis or nuclear magnetic resonance spectroscopy.

A variety of suitable compounds have been discussed above. To test the function of the NCAM binding molecule, the inventors have established a simple cell culture system (aggregate cell cultures) that permits a quantitative evaluation of the effect of various ligands. Hippocampal cells are provided from rat embryos. The cells are grown in a defined medium and dissociated cells are seeded in microtiter plates. After 24 h, the amount of aggregates are counted. Compounds to be tested are added to the cell suspension immediately before seeding of the cells in micro-wells. When NCAM binding compounds are present during the aggregation of cells, smaller, but more numerous cell aggregates are seen when quantified 24 h after seeding of the cells. The inhibiting effect of the ligands results in a blockage of the formation of large aggregates from many small aggregates as the adhesion properties of NCAM are blocked. Thus, small but more numerous cell aggregates are seen in the presence of active ligands. Such an effect is due to the presence of different compounds of the invention during the aggregation of cells. The system allows for the examination of disaggregation of the treated cells.

The invention also relates to a pharmaceutical composition comprising one or more of the compounds as defined above. The compounds are preferably formulated as dimers or multimers such as discussed above. In the present context the term pharmaceutical composition is used synonymously with the term medicament.

The scope of the invention is further related to a pharmaceutical composition capable of preventing death of cells in vitro or in vivo, wherein the composition is administered to a subject, in vitro or in vivo in an effective amount of one or more of the compounds described above or a composition as described below, so as to promote cell differentiation and modulation of proliferation of neural cells and neuronal plasticity, and stimulation of survival and regeneration of NCAM presenting cells and/or NCAM ligand presenting cells in several tissues and organs as discussed herein. The medicament of the invention comprises an effective amount of one or more of the compounds as defined above, or a composition as defined above in combination with pharmaceutically acceptable additives. Such medicament may suitably be formulated for oral, percutaneous, intramuscular, intravenous, intracranial, intrathecal, intracerebroventricular, intranasal or pulmonal administration.

The present invention further concerns a medicament for the treatment of diseases and conditions of the central and peripheral nervous system, of the muscles or of various organs, wherein said medicament comprises an effective amount of one or more of the compounds as defined above or a composition as defined above in combination with pharmaceutically acceptable additives or carriers. Such medicament may suitably be formulated for oral, percutaneous, intramuscular, intravenous, intracranial, intrathecal, intracerebroventricular, intranasal or pulmonal administration.

For most indications a localised or substantially localised application is preferred. The compounds are in particular used in combination with a prosthetic device such as a prosthetic nerve guide. Thus, in a further aspect, the present invention relates to a prosthetic nerve guide, characterised in that it comprises one or more of the compounds or the composition defined above. Nerve guides are known in the art.

In connection with the use in nerve guides, the administration may be continuous or in small portions based upon controlled release of the active compound(s). Furthermore, precursors may be used to control the rate of release and/or site of release. Other kinds of implants and well as oral administration may similarly be based upon controlled release and/or the use of precursors.

Strategies in formulation development of medicaments and compositions based on the compounds of the present invention generally correspond to formulation strategies for any other protein-based drug product. Potential problems and the guidance required to overcome these problems are dealt with in several textbooks, e.g. “Therapeutic Peptides and Protein Formulation. Processing and Delivery Systems”, Ed. A. K. Banga, Technomic Publishing AG, Basel, 1995.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH buffering agents, which enhance the effectiveness or transportation of the preparation.

Formulations of the compounds of the invention can be prepared by techniques known to the person skilled in the art. The formulations may contain pharmaceutically acceptable carriers and excipients including microspheres, liposomes, micro-capsules, nanoparticles or the like.

The preparation may suitably be administered by injection, optionally at the site, where the active ingredient is to exert its effect. Additional formulations which are suitable for other modes of administration include suppositories, nasal, pulmonal and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

Other formulations are such suitable for nasal and pulmonal administration, e.g. inhalators and aerosols.

The active compound may be formulated as neutral or salt forms. Pharmaceutically acceptable salts include acid addition salts (formed with the free amino groups of the peptide compound) and which are formed with inorganic acids such as, for example, hydrochloric or phosphoric acids, or such organic acids as acetic acid, oxalic acid, tartaric acid, mandelic acid, and the like. Salts formed with the free carboxyl group may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The preparations are administered in a manner compatible with the dosage formulation and in such amount as will be therapeutically effective. The quantity to be administered depends on the subject to be treated, including, e.g. the weight and age of the subject, the disease to be treated and the stage of disease. Suitable dosage ranges are of the order of several hundred μg active ingredient per administration with a preferred range of from about 0.1 μg to 100 mg, such as in the range of from about 1 μg to 100 mg, and especially in the range of from about 10 μg to 50 mg. Administration may be performed once or may be followed by subsequent administrations. The dosage will also depend on the route of administration and will vary with the age and weight of the subject to be treated. A preferred dosis would be in the interval 0.5 mg to 50 mg per 70 kg body weight.

Some of the compounds of the present invention are sufficiently active, but for others, the effect will be enhanced if the preparation further comprises pharmaceutically acceptable additives and/or carriers. Such additives and carriers will be known in the art. In some cases, it will be advantageous to include a compound, which promote delivery of the active substance to its target.

In another embodiment it may be advantageous to administer the compound(s) according to the invention with other substances to obtain a synergistic effect. Examples of such other substances may be a growth factor, which can induce differentiation, or a hormone, or a transplant of cells, including a transplant of stem cells, or gene therapy, or immunotherapy.

These mentioned compounds and compositions may be used to treat conditions effecting the peripheral and/or the central nervous system and/or muscles and other tissues expressing NCAM or NCAM ligands as well as other conditions in which a stimulation of NCAM function or the function of a NCAM ligand is beneficial.

In one aspect of the invention treatment by the use of the compounds according to the invention is useful for the stimulation of regenerating cells which are degenerating or at risk of dying due to a variety of factors, such as traumas and injuries, acute diseases, chronic diseases and/or disorders, in particular degenerative diseases normally leading to cell death, other external factors, such as medical and/or surgical treatments and/or diagnostic methods that may cause formation of free radicals or otherwise have cytotoxic effects, such as X-rays and chemotherapy. In relation to chemotherapy the NCAM binding comppunds according to the invention are useful in cancer treatment.

Also, the compounds according to the invention may be used for preventing cell death of cells being implanted or transplanted. This is particularly useful when using compounds having a long term effect.

In another aspect of the invention the compounds may be synthesised and secreted from implanted or injected gene manipulated cells.

Thus, the treatment comprises the use of said compound for diseases or conditions of the central and peripheral nervous system, such as postoperative nerve damage, traumatic nerve damage, impaired myelination of nerve fibers, postischaemic damage, e.g. resulting from a stroke, Parkinson's disease, Alzheimer's disease, Huntington's disease, dementias such as multiinfarct dementia, sclerosis, nerve degeneration associated with diabetes mellitus, disorders affecting the circadian clock or neuro-muscular transmission, and schizophrenia, mood disorders, such as manic depression; for treatment of diseases or conditions of the muscles including conditions with impaired function of neuromuscular connections, such as after organ transplantation, or such as genetic or traumatic atrophic muscle disorders; or for treatment of diseases or conditions of various organs, such as degenerative conditions of the gonads, of the pancreas such as diabetes mellitus type I and II, of the kidney such as nephrosis and of the heart, liver and bowel.

Also, the present compound may be used in relation to diseases or conditions of the muscles including conditions with impaired function of neuro-muscular connections, such as genetic or traumatic atrophic muscle disorders; or for the treatment of diseases or conditions of various organs, such as degenerative conditions of the gonads, of the pancreas, such as diabetes mellitus type I and II, of the kidney, such as nephrosis the compounds according to the invention may be used for preventing cell death, i.e. stimulating survival.

Furthermore, the compound and/or pharmaceutical composition may be for preventing cell death of heart muscle cells, such as after acute myocardial infarction, or after angiogenesis. Furthermore, in one embodiment the compound and/or pharmaceutical composition is for the stimulation of the survival of heart muscle cells, such as survival after acute myocardial infarction. In another aspect the compound and/or pharmaceutical composition is for revascularisation, such as after injuries.

In another aspect the compound and/or pharmaceutical composition is used for the stimulation of the ability to learn and/or of the short and/or long term memory.

Furthermore, the compound of the invention and/or a fragment thereof may be for the manufacture of a medicament for treatment of normal, degenerated or damaged NCAM and/or NCAM ligand presenting cells.

In particular the compound and/or pharmaceutical composition of the invention may be used in the treatment of clinical conditions, such as Neoplasms such as malignant neoplasms, benign neoplasms, carcinoma in situ and neoplasms of uncertain behavior, diseases of endocrine glands, such as diabetes mellitus, psychoses, such as senile and presenile organic psychotic conditions, alcoholic psychoses, drug psychoses, transient organic psychotic conditions, Alzheimer's disease, cerebral lipidoses, epilepsy, general paresis [syphilis], hepatolenticular degeneration, Huntington's chorea, Jakob-Creutzfeldt disease, multiple sclerosis, Pick's disease of the brain, syphilis, Schizophrenic disorders, affective psychoses, neurotic disorders, personality disorders, including character neurosis, nonpsychotic personality disorder associated with organic brain syndromes, paranoid personality disorder, fanatic personality, paranoid personality (disorder), paranoid traits, sexual deviations and disorders, mental retardation, disease in the nervesystem and sense organs, cognitive anomalies, inflammatory disease of the central nervous system, such as meningitis, encephalitis, Cerebral degenerations such as Alzheimer's disease, Pick's disease, senile degeneration of brain, communicating hydrocephalus, obstructive hydrocephalus, Parkinson's disease including other extra pyramidal disease and abnormal movement disorders, spinocerebellar disease, cerebellar ataxia, Marie's, Sanger-Brown, Dyssynergia cerebellaris myoclonica, primary cerebellar degeneration, such as spinal muscular atrophy, familial, juvenile, adult spinal muscular atrophy, motor neuron disease, amyotrophic lateral sclerosis, motor neuron disease, progressive bulbar palsy, pseudobulbar palsy, primary lateral sclerosis, other anterior horn cell diseases, anterior horn cell disease, unspecified, other diseases of spinal cord, syringomyelia and syringobulbia, vascular myelopathies, acute infarction of spinal cord (embolic) (nonembolic), arterial thrombosis of spinal cord, edema of spinal cord, subacute necrotic myelopathy, subacute combined degeneration of spinal cord in diseases classified elsewhere, myelopathy, drug-induced, radiation-induced myelitis, disorders of the autonomic nervous system, disorders of peripheral autonomic, sympathetic, parasympathetic, or vegetative system, familial dysautonomia [Riley-Day syndrome], idiopathic peripheral autonomic neuropathy, carotid sinus syncope or syndrome, cervical sympathetic dystrophy or paralysis, peripheral autonomic neuropathy in disorders classified elsewhere, amyloidosis, diseases of the peripheral nerve system, brachial plexus lesions, cervical rib syndrome, costo-clavicular syndrome, scalenus anterior syndrome, thoracic outlet syndrome, brachial neuritis or radiculitis, including in newborn. Inflammatory and toxic neuropathy, including acute infective polyneuritis, Guillain-Barre syndrome, Postinfectious polyneuritis, polyneuropathy in collagen vascular disease, disorders affecting multiple structures of eye, purulent endophthalmitis, diseases of the ear and mastoid process, chronic rheumatic heart disease, ischaemic heart disease, arrhythmia, diseases in the pulmonary system, abnormality of organs and soft tissues in newborn, including in the nerve system, complications of the administration of anesthetic or other sedation in labor and delivery, diseases in the skin including infection, insufficient circulation problem, injuries, including after surgery, crushing injury, burns. Injuries to nerves and spinal cord, including division of nerve, lesion in continuity (with or without open wound), traumatic neuroma (with or without open wound), traumatic transient paralysis (with or without open wound), accidental puncture or laceration during medical procedure, injury to optic nerve and pathways, optic nerve injury, second cranial nerve, injury to optic chiasm, injury to optic pathways, injury to visual cortex, unspecified blindness, injury to other cranial nerve(s), injury to other and unspecified nerves. Poisoning by drugs, medicinal and biological substances, genetic or traumatic atrophic muscle disorders; or for the treatment of diseases or conditions of various organs, such as degenerative conditions of the gonads, of the pancreas, such as diabetes mellitus type I and II, of the kidney, such as nephrosis.

More particular for the treatment of diseases or conditions of the central and peripheral nervous system, such as postoperative nerve damage, traumatic nerve damage, impaired myelination of nerve fibers, postischaemic damage, e.g. resulting from a stroke, Parkinson's disease, Alzheimer's disease, Huntington's disease, dementias such as multiinfarct dementia, sclerosis, nerve degeneration associated with diabetes mellitus, disorders affecting the circadian clock or neuro-muscular transmission, and schizophrenia, mood disorders, such as manic depression; for treatment of diseases or conditions of the muscles including conditions with impaired function of neuro-muscular connections, such as after organ transplantation, or such as genetic or traumatic atrophic muscle disorders; or for treatment of diseases or conditions of various organs, such as degenerative conditions of the gonads, of the pancreas such as diabetes mellitus type I and II, of the kidney such as nephrosis and of the heart and bowel, and for the treatment of postoperative nerve damage, traumatic nerve damage, impaired myelination of nerve fibers, postischaemic, e.g. resulting from a stroke, Parkinson's disease, Alzheimer's disease, dementias such as multiinfarct dementia, sclerosis, nerve degeneration associated with diabetes mellitus, disorders affecting the circadian clock or neuro-muscular transmission, and schizophrenia, mood disorders, such as manic depression.

It is also within the scope of the invention to use the compound and/or pharmaceutical composition for the promotion of wound-healing. The present compounds are capable of interfering with cell adhesion and thereby promote the wound healing process.

The invention further discloses the use of the compound and/or pharmaceutical composition in the treatment of cancer. NCAM regulates motility and inhibits cancer cells from spreading.

In a further embodiment of the invention the use of the compound and/or pharmaceutical composition is for stimulation of the ability to learn and/or of the short and/or long term memory.

In many instances, it will be necessary to administrate the formulation multiple times. Administration may be a continuous infusion, such as intraventricular infusion or administration in more doses such as more times a day, daily, more times a week, or weekly. It is preferred that administration of the medicament is initiated before or shortly after the individual has been subjected to the factor(s) that may lead to cell death. Preferably the medicament is administered within 8 hours from the factor onset, such as within 5 hours from the factor onset. Many of the compounds exhibit a long term effect whereby administration of the compounds may be conducted with long intervals, such as 1 week or 2 weeks.

In one embodiment of the invention the administration of the present compound may be immediately after an acute injury, such as an acute stroke, or at the most 8 hours after said stroke in order for the present compound to have a stimulatory effect on cell survival. Further, in cases concerning proliferation and/or differentiation the administration according to the invention is not time dependent, i.e. it may be administered at any time.

In another aspect the invention relates to a process of producing a pharmaceutical composition, comprising mixing an effective amount of one or more of the compounds of the invention, or a pharmaceutical composition according to the invention with one or more pharmaceutically acceptable additives or carriers, and administer an effective amount of at least one of said compound, or said pharmaceutical composition to a subject.

In one embodiment the process as mentioned above, the compounds are used in combination with a prosthetic device, wherein the device is a prosthetic nerve guide.

In a further aspect of the process the prosthetic nerve guide is characterised in that it comprises at least one compound, or a pharmaceutical composition as defined by the invention.

A further aspect of the present invention relates to the use of an NCAM (neural cell adhesion molecule) binding compound capable of stimulating NCAM signalling and/or interfering with cell adhesion. In one embodiment of the invention the use of a compound and/or pharmaceutical composition is for the manufacture of a medicament. Such use may be of any of the compounds of the invention.

The use of said compound may in one embodiment be for the manufacture of a medicament for the treatment of normal, degenerated or damaged NCAM presenting cells.

The invention also discloses the use, wherein said compound is for the manufacture of a medicament for the treatment comprising the stimulation of differentiation of NCAM presenting cells and/or survival thereof.

In one aspect of the invention the use is for the manufacture of a medicament comprising treatment of diseases and conditions of the central and peripheral nervous system, or of the muscles or of various organs as discussed above.

In yet a further aspect the invention relates to a method of treating an individual suffering from one or more of the diseases discussed above by administering the said individual a compound as described herein or a pharmacautical composition comprising said compound.

### EXPERIMENTALS

The following are non-limiting illustrations of the present invention.

Materials

Fura-2-AM, fura-2 pentapotassium salt and CaEGTA/K₂EGTA buffers were obtained from Molecular Probes (Eugene, Oreg., USA). The calcium channel antagonists ω-conotoxin MVIIA and nifedipine were obtained from Alomone Labs (Jerusalem, Israel). Rabbit FGF-R antiserum (1:1000) raised against a synthetic peptide corresponding to amino acids 119-144 of the chicken FGF-R situated close to the so-called CAM homology domain and the P38 MAP kinase inhibitor SB203580 were from Upstate Biotechnology (Lake Placid, N.Y., USA). The MEK inhibitor PD98059 was from New England Biolabs (Beverly, Mass., USA). The presumed inhibitor of intracellular calcium mobilisation TMB-8 (8-(N,N-diethylamino)octyl-3,4,5-trimethoxybenzoate hydrochloride) and the Src family tyrosine kinase inhibitor PP1 were obtained from Calbiochem (La Jolla, Calif., USA). TentaGel resin was obtained from Rapp Polymere (Tübirigen, Germany). Rink amide linker and Fmoc-protected amino acids were obtained from Novabiochem (Läufelfingen, Switzerland). DMEM, EDTA and B27 were obtained from Gibco BRL (Paisley, Scotland). Plastic ware for cell culture was obtained from NUNC A/S (Roskilde, Denmark). All other reagents were obtained from Sigma (St Louis, Mo., USA). NCAM from Postnatal day 10 rat brain was purified as previously described (RAsmussen et al., 1982)(Krog et al., 1992).

Cell Culture

Fibroblastoid L929-cells, stably transfected with either NCAM-140 or empty vector, were grown in Dulbecco's Modified Eagles Medium (DMEM) supplemented with 10% fetal bovine serum (FBS), penicillin (100 U/ml) and streptomycin (100 μg/ml) in a humidified atmosphere at 37° C. and 5% CO₂. For establishment of monolayers for cocultures, L-cells were dislodged with trypsin (0.5 mg/ml) and EDTA (0.75 mM), seeded at a density of approximately 55,000 cells/cm² in 4 or 8 well LabTek chamber slides with a growth surface of plastic coated with fibronectin and grown for 24 hours.

Dissociated hippocampal cells prepared from rat embryos gestational day 18 were seeded in microwell plates 50,000 cells in 15 μl of medium per well as previously described (Maar et al., 1997)(Rønn et al., 1999). All animals were handled in accordance with the national guidelines for animal welfare. Cells were grown in Neurobasal medium supplemented with B27, 20 mM HEPES, penicillin (100 U/ml), streptomycin (100 μg/ml) and 0.4% w/v bovine serum albumin. After 24 h in culture, the number of aggregates per well was determined. For analysis of neurite outgrowth, 5,000 cells/well were seeded in 8 well LabTek Tissue Culture Chamber Slides with a growth surface of Permanox plastic. After 24 h, images of neurons were captured and analysed by means of computer-assisted microscopy as described (Rønn et al., 2000).

For cocultures, primary hippocampal neurons were seeded on monolayers of fibrobroblasts in Neurobasal medium supplemented with 2% v/v FBS. After 24 h, cultures were fixed and stained for GAP-43 immunoreactivity for selective visualisation of neurons as previously described (Skladchikova et al., 1999).

The PC12-E2 cell line was a generous gift from Dr. Klaus Seedorf, Hagedorn Research Institute, Denmark. Cells were grown in DMEM supplemented with 5% v/v FBS and 10% v/v horse serum (HS). For calcium imaging, PC12E2 cells were dislodged mechanically by tapping and seeded at a density of 5-30,000 cells/cm² in 4 or 8 well LabTek chambered cover-slides (NUNC) with a growth surface of plastic coated with fibronectin (10 μg/ml) and grown for 1 to 5 days. In some cases, neuronal differentiation was induced by changing the medium to DMEM supplemented with 1% v/v FBS, 1% v/v HS and NGF (50 ng/ml) or FGF-2 (10 ng/ml).

Calcium Imaging

Cells were washed in HANK's solution comprising KCl (5.4 mM), NaCl (137 mM), NaHCO₃ (2 mM), MgSO₄ (0.8 mM), Na₂HPO₄ (0.27 mM), Glucose (5.6 mM), CaCl₂ (1.25 mM), KH₂PO₄ (0.44 mM) and loaded with fura-2 acetomethyl ester (Fura-2 AM, 2 μM, Molecular Probes, Eugene, Oreg., USA) dissolved in Dimethyl Sulfoxide (DMSO) for 35 min at 20° C. in the dark. Hereafter, cells were washed four times and placed on the stage of an inverted Axiovert 100 TV microscope (Zeiss, Göttingen, Germany) equipped with an oil immersion UV objective (Zeiss Fluar 40×, 1.3 numerical aperture). Imaging was performed using a Sensicam 12 bit cooled CCD camera (PCO, Keilheim, Germany) and a J&M monochromator (J&M; Aalen, Germany). The software Imaging Workbench (Axon, Foster City, Calif., USA) was used for data acquisition and analysis. Ratio-images were obtained after background subtraction from images collected at wavelengths over 510 nm after excitation at 340 and 380 nm respectively at sampling rates between 0.1 and 1 Hz. Calibration was performed using CaEGTA/K₂EGTA buffers with known concentrations of free calcium and fura-2 pentapotassium salt (5 μM). The concentration of free calcium was estimated according to the formula, [Ca²⁺]ᵢ=K_{d}·(R−Rₘᵢₙ)/(Rₘₐₓ−R)·(F380ₘₐₓ/F380ₘᵢₙ), where R is the ratio of background subtracted fluorescence intensities obtained at excitation at 340 and 380 nm respectively, Rₘₐₓ is the ratio at saturating calcium, Rₘᵢₙ is the ratio at zero free calcium, F380ₘᵢₙ is the intensity at saturating free calcium exciting at 380 nm, while F380ₘₐₓ is the intensity at zero free calcium. Rₘₐₓ was determined in situ using Fura-2 AM loaded cells in the presence of ionomycin (5 μM) and high extracellular calcium (10 mM). Values determined were: Rₘₐₓ 9.0; Rₘᵢₙ 0.68; F380ₘₐₓ/F380ₘᵢₙ 7.0. The K_{d} used was 236 nM (Groden et al., 1991). Peptides to be tested were applied directly to the cell culture chambers in a volume corresponding to half of the volume present in the chamber prior to application to ensure an even distribution.

Synthesis of Peptides

Peptides were synthesised on TentaGel resin. All peptides had an alanine N-terminally.

NMR Spectroscopy

Two samples were used in the characterization of the P10 binding site on NCAM IgI: (A) ¹⁵N labeled IgI 0.025 mM (B) a mixture of ¹⁵N labeled IgI 0.025 mM and 0.2 mM P10d-peptide. Both samples were prepared in 90% H2O:10% D2O, 150 mM NaCl, 5 mM sodium phosphate, 0.02% sodium azide, pH 7.34 buffer. Two ¹⁵N-HSQC NMR spectra were recorded with 12000 Hz spectral width, 3792 complex points in t₂ and 170 increments in t₁ on a Varian Unity Inova 800 MHz spectrometer at 298K (42). The transformation and analysis of spectra were performed using the MNMR and PRONTO computer programs, respectively (43).

Peptides

Peptides having the sequences NBP10 (SEQ ID NO:1=AKKMWKKTW) and NBP9 (AWKEASWK) which both contained lysines flanked by tryptophans were used in the examples. It has been reported that multimeric forms of peptide ligands identified by means of phage display peptide libraries have a higher potency for receptor activation than moriomeric forms (Lam et al., 1991). Therefore, the peptides were synthesised both as monomers and as dendrimers composed of four monomers coupled to a lysine backbone, or 20-mers coupled to bovine serum albumin (BSA) in order to compare the effects of monomeric and multimeric ligands.

### EXAMPLE 1

The NBP10-Peptide Inhibits Cell Aggregation

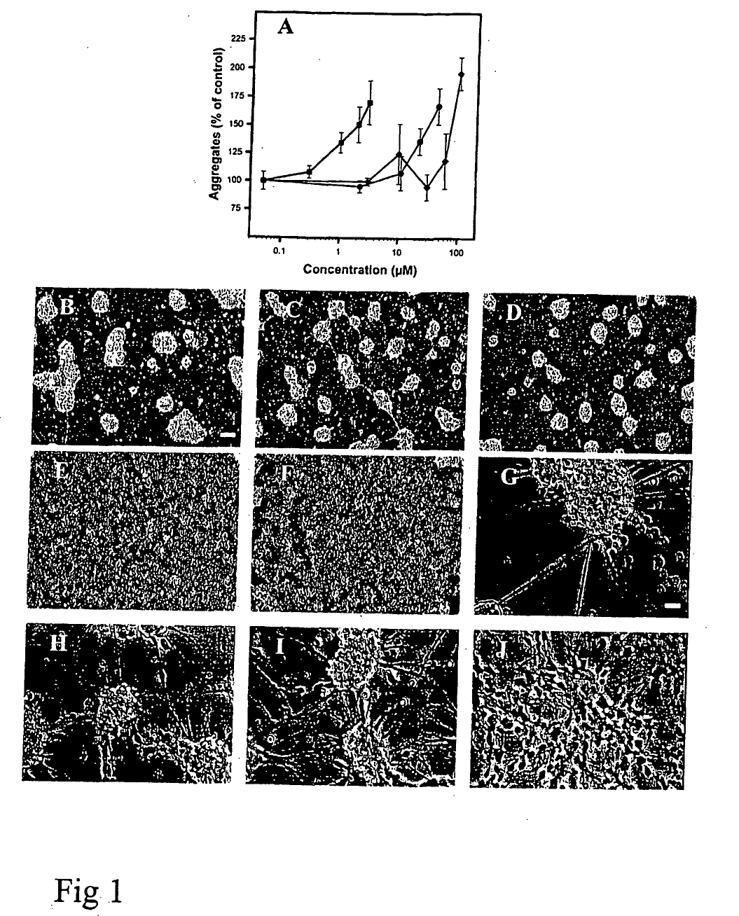

An important function of NCAM is mediation of cell adhesion. In order to select functional ligands of NCAM from the identified peptide sequences the ability of the peptides to inhibit cell aggregation between hippocampal cells in primary cultures of hippocampal neurons grown under conditions permitting cell aggregation (Maar et al., 1997) was tested. In this model system, recombinant NCAM modules or antibodies recognising NCAM have been shown to inhibit cell aggregation potently (Maar et al., 1997)(Kiselyov et al., 1997)(4349). When tested as a BSA-bound multimeric peptide, the NBP10-peptide resulted in the formation of smaller but more numerous aggregates indicating an inhibition of cell adhesion (FIG. 1. A). NBP10 inhibited cell aggregation both in monomeric and in dendrimeric form, the dendrimeric form being the most potent. The effect of NBP10 on cell aggregation was only quantified at low concentrations of the peptide (FIG. 1CD). High peptide concentrations changed the morphology of the cultures dramatically leading to the formation of a network of single cells or very small clusters of cells interconnected by numerous thin processes (FIG. 1EF). Under these conditions, the number of aggregates could not be quantified. Thus, the NBP10 monomer (NBP10m) induced the formation of small but distinct aggregates in a concentration of 60 μM (FIG. 1C,I) whereas cell cultures grown in the presence of NBP10m in a concentration of 200 μM had a very different morphology without distinct cell aggregates (FIG. 1E,J). Similarly, the NBP10 dendrimer (NBP10) induced the formation of small but distinct aggregates in a concentration of 1 or 2 μM (FIG. 1D,H) whereas cell cultures grown in the presence of NBP10d in a concentration of 6 μM had no distinct cell aggregates (FIG. 1F).

These findings showed that the NBP10-peptide interfered with cell adhesion, a well-known NCAM function. The peptide dendrimer was most potent with comparable effects at a concentration approximately 50 times lower than that used of the peptide monomer.

### EXAMPLE 2

NBP10 Modulates Neurite Outgrowth Induced by Homophilic NCAM-Binding

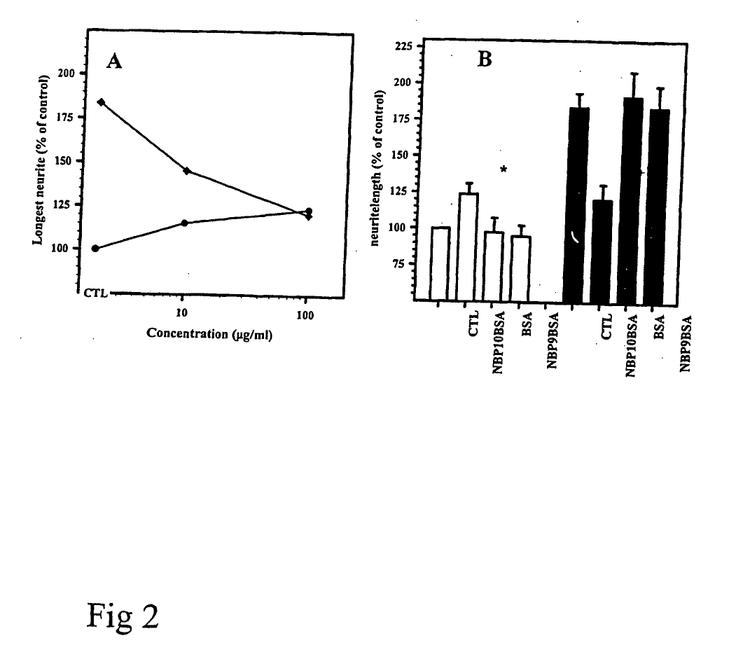

To investigate the effect of the NBP10 peptide on NCAM-mediated cell adhesion, the neurite outgrowth induced by homophilic NCAM binding in cocultures of NCAM-140 transfected fibroblasts and primary hippocampal neurons was studied. It has previously been shown that under these conditions, neurite outgrowth is stimulated specifically by homophilic NCAM binding when neurons are grown on monolayers of NCAM-expressing fibroblasts when compared to cultures in which neurons are grown on monolayers of fibroblasts without NCAM-expression (Williams et al., 1994)(4180). In the present study, a similar stimulation of neurite outgrowth induced by homophilic NCAM binding was observed. This stimulation was inhibited in a dose-dependent manner by the BSA-coupled NBP10-peptide (FIG. 2) in concentrations similar to those found to inhibit cell cell aggregation (FIG. 1) whereas NBP9-BSA or BSA alone had no effect indicating that NBP10-BSA interfered specifically with homophilic NCAM-binding thereby preventing NCAM-induced neurite outgrowth without interfering with basal neurite outgrowth. When neurons were maintained on fibroblasts without NCAM-expression, a small stimulation of neurite outgrowth of approx. 25% by NBP10-BSA was observed (FIG. 2). Under these conditions, neurite outgrowth depends on recognition events other than homophilic NCAM-interactions including integrin-binding and, possibly, heterophilic NCAM-interactions. Hence, when NCAM on the neurons is not engaged in homophilic binding, the NBP10-peptide may bind to NCAM and thereby stimulate outgrowth of neurites although not as efficiently as the NCAM molecule itself when presented by a fibroblast monolayer.

### EXAMPLE 3

Stimulation of Neurite Outgrowth in Dissociated Cultures of Primary Hippocampal Neurons by NBP10

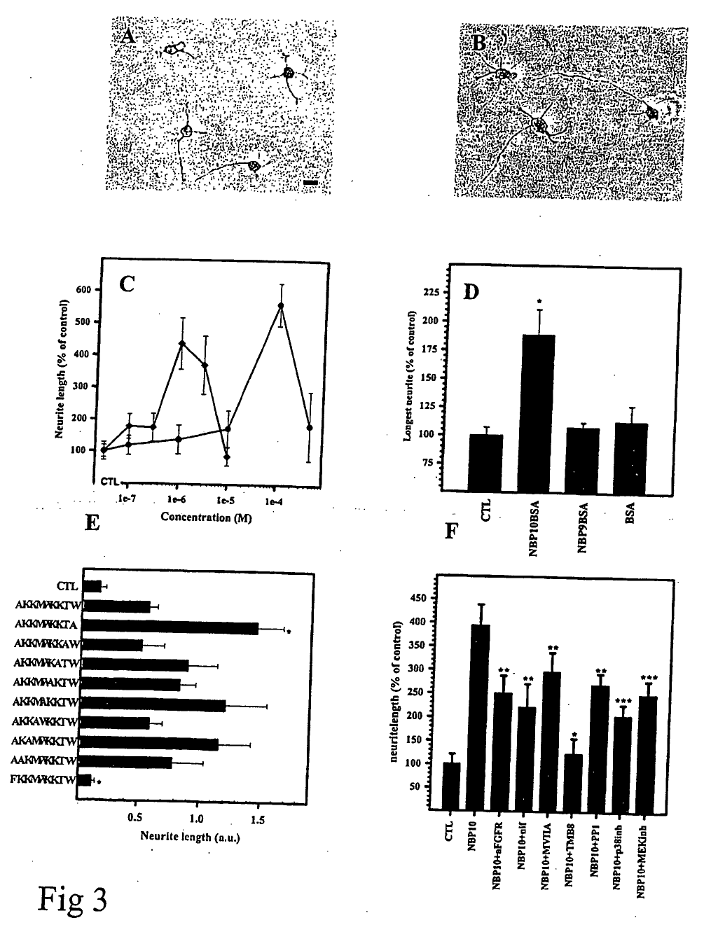

The effect of NBP10 in dissociated cultures of primary hippocampal neurons grown on a plastic substratum were examined. Under these conditions, the dendrimeric NBP10-peptide (NBP10d) had a strong stimulatory effect on neurite outgrowth in a concentration of 1 μM (FIG. 3AB). The monomeric NBP10 peptide (NBP10m) also stimulated neurite outgrowth although with much lower potency than NBP10d (FIG. 3C). However the maximal effect of NBP10d and NBP10m was at the same level and both exhibited a bell shaped dose-response relationship with a maximal effect at concentrations around 1 μM (NBP10d) and 100 μM (NBP10m) respectively. The BSA-coupled NBP10 peptide (NBP10BSA) stimulated neurite outgrowth at a concentration of 22 μM while NBP9BSA and BSA alone had no effect (FIG. 3D).

The effect of single substitutions in the monomeric NBP10-sequence (FIG. 3E) was tested. Alanine substitution of the amino acid residue W9 resulted in a statistically significant increase in neurite outgrowth while a phenylalanine substitution to the amino acid residue A1 resulted in a significant inhibition of the effect. This indicates that the amino acid residues A1 and W9 probably are of importance for the observed neuritogenic effect of the NBP10-peptide.

### EXAMPLE 4

NBP10 Induces NCAM-Dependent Signal Transduction

The involvement of presumed NCAM-dependent signal transduction pathways in neurite outgrowth induced by the NBP10-peptide was addressed The NBP10-dendrimer had its maximal effect at a concentration of 1 μM while the monomer had a comparable maximal effect at a concentration of 1.00 μM (FIG. 1C) indicating that NCAM-clustering by the multimeric dendrimer peptide may increase the neuritogenic effect of the NBP10-peptide. The dose-response relationship was bell-shaped similarly to findings for a recently reported synthetic peptide ligand of the NCAM IgI module, C3 (Rønn et al., 1999), and for fibroblast growth factor-2 (FGF-2) and arachidonic acid (AA), which are both believed to be downstream components of an NCAM-dependent signalling pathway (Doherty and Walsh, 1996). Thus, it has been suggested that neurite outgrowth induced by NCAM-NCAM-binding depends on an interaction of NCAM with FGF-receptor (FGF-R) followed by activation of a signalling pathway leading to an increase in the intracellular concentration of calcium (Williams et al., 1994)(Doherty and Walsh, 1996). In addition, a p59^{fyn}-dependent activation of the Ras-MAP-kinase pathway has been implicated in NCAM-induced neurite outgrowth (Schmid et al., 1999)(Kolkova et al., 2000). Whether NBP10-induced neurite outgrowth might depend on an activation of a similar signal transduction pathway by testing the effect of a number of compounds previously reported to inhibit NCAM-dependent signalling was investigated (FIG. 3F). It was observed that the neurite outgrowth response of the NBP10-peptide was partially inhibited by an antibody previously shown to inhibit specifically neurite outgrowth induced by homophilic NCAM-binding (Williams et al., 1994) indicating that the neuritogenic effect of NBP10 may be mediated by an NCAM-dependent activation of an FGF-R dependent signalling pathway. NBP10-induced neurite outgrowth also was partially inhibited by PP1, an inhibitor of p59^{fyn} and other Src family tyrosine kinases, by PD98059, an inhibitor of MEK, and SB203580, an inhibitor of P38MAP kinase, indicating an involvement of the Ras-MAP-kinase signalling pathway previously shown to be necessary for NCAM-dependent neurite outgrowth (Kolkova et al., 2000). Although some of the inhibitors used here may have undesired side effects, there is a striking correlation between their inhibitory effect on NBP10-induced neurite outgrowth and their previously reported inhibitory effect on NCAM-induced neurite outgrowth indicating that NBP10 induces neurite outgrowth specifically through NCAM-binding.

The effect of antagonists of calcium mobilisation from intra- and extracellular calcium stores was tested and a partial inhibition of NBP10-induced neurite outgrowth by nifedipine, an L-type VDCC antagonist, ω-conotoxin MVIIA, an N-type VDCC antagonist and a complete inhibition by TMB-8, a presumed inhibitor of intracellular calcium mobilisation was observed. This suggests that a calcium influx through plasma membrane VDCCs is involved in NBP10-induced neurite outgrowth in accordance with previous observations for neurite outgrowth induced by homophilic NCAM-binding in coculture models (Doherty et al., 1991). However, the present finding that NBP10-induced neurite outgrowth is inhibited by TMB-8 indicates that mobilisation of calcium from intracellular calcium stores probably also is involved in NCAM-dependent neurite outgrowth.

### EXAMPLE 5

NBP10 Increases Intracellular Calcium in PC12E2 Cells

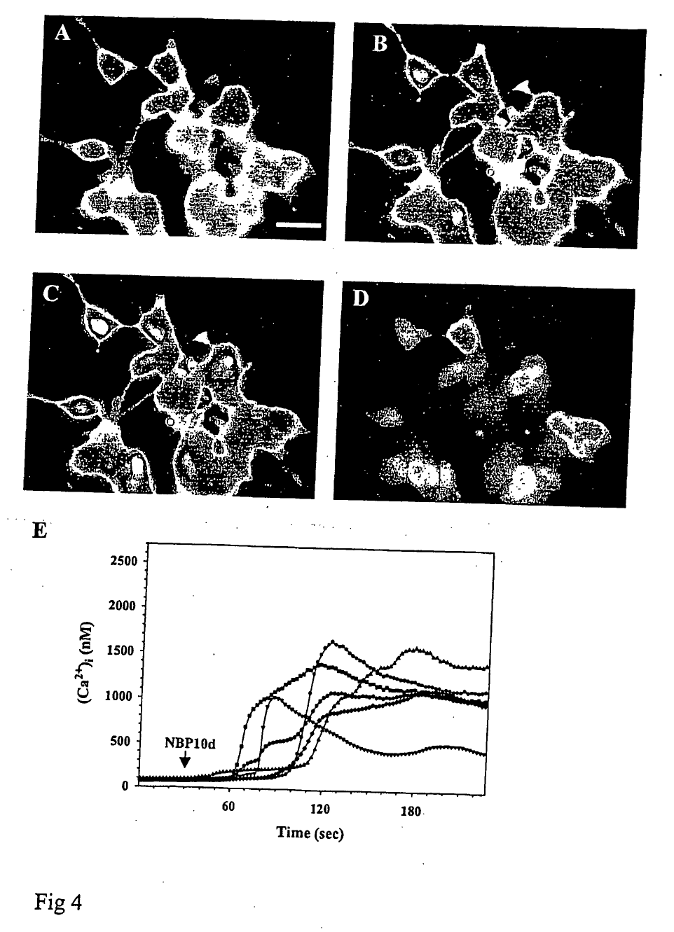

Since NBP10-induced neurite outgrowth appeared to depend on VDCCs and intracellular calcium stores, it was tested whether NBP10 was capable of directly influencing intracellular calcium in neuronal cells. When NBP10-d was applied in a concentration of 50 μM to fura-2 loaded PC12E2 cells, a sustained increase in intracellular calcium was observed as evidenced by an increase in the fluorescence following excitation at a wavelength of 340 nm and the concomitant decrease in the fluorescence recorded following excitation at 380 nm as shown in FIG. 4. When lower concentrations of NBP10 were applied to PC12E2 cells only a fraction of the cells showed detectable increases in intracellular calcium (not shown). These observations further support the hypothesis that signal transduction and the subsequent neurite outgrowth response induced by NBP10-peptide and NCAM rely on an increased intracellular calcium.

### EXAMPLE 6

NBP10 Binds the NCAM Ig1 Module

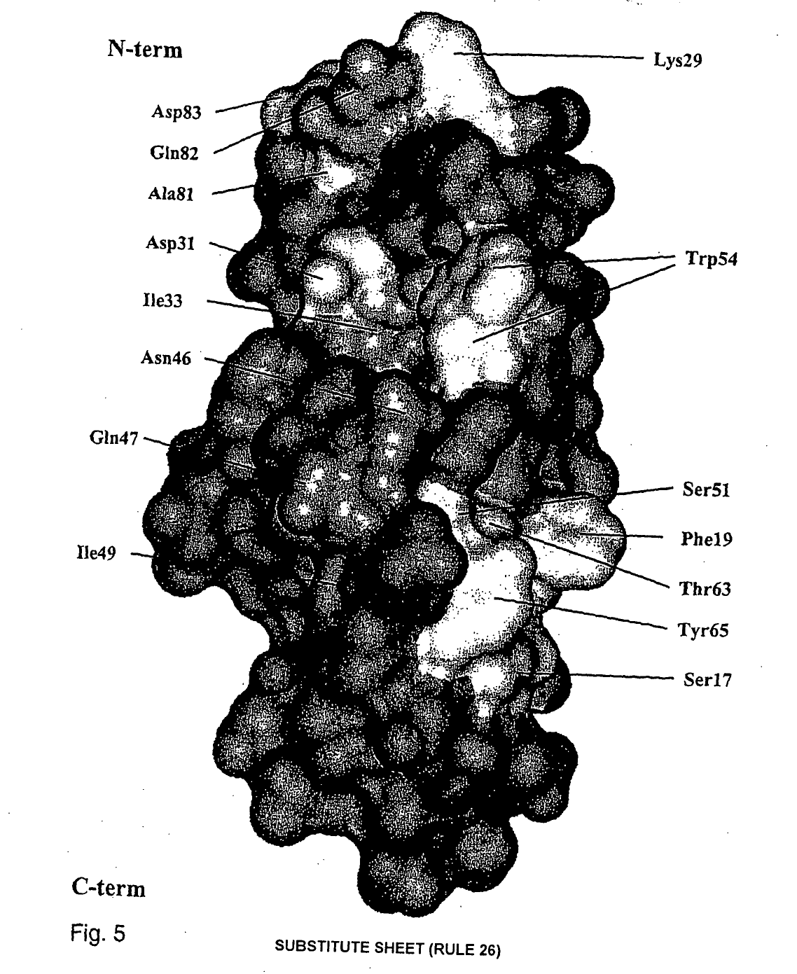

By nuclear magnetic resonance analysis (NMR), the NBP10 peptide dendrimer was found to bind the recombinant Ig1 module of NCAM (FIG. 5). Upon binding of NBP10d, the amino acid residues shown in the figure exhibited chemical shifts.

### REFERENCES

Bodenhosen, G. and Ruben, D. J. (1980) Chem. Phys. lett. 69, 185-189.
Cole, G. J. and Glaser, L. (1986) Journal of Cell Biology 102, 403-412. Doherty, P. and Walsh, F. S. (1996) Molecular and Cellular Neuroscience 8, 99-111.
Doherty, P. and Walsh, F. S. (1996) Molecular & Cellular Neurosciences 8, 99-111.
Doherty, P., Ashton, S. V., Moore, S. E., and Walsh, F. S. (1991) Cell 67, 21-33.
Holley, J. A. and Yu, R. K. (1987) Developmental Neuroscience 9, 105-119.
Fransen, E., D'Hooge, R., Van Camp, G., Verhoye, M., Sijbers, J., Reyniers, E., Soriano, P., Kamiguchi, H., Willemsen, R., Koekkoek, S. K., De Zeeuw, C. I., De, Deyn, P. P., Van der Linden, A., Lemmon, V., Kooy, R. F., and Willems, P. J. (1998) Human Molecular Genetics 7, 999-1009.
Furka, A., Sebestyen, F., Asgedom, M., and Dibo, G. (1991) International Journal of Peptide & Protein Research 37, 487-493.
Jensen, P. H., Soroka, V., Thomsen, N. K., Ralets, I., Berezin, V., Bock, E., and Poulsen, F. M. (1999) Nature Structural Biology 6 no. 5, 486-493.
Groden, D. L., Guan, Z., and Stokes, B. T. (1991) Cell Calcium 12, 279-287.
Kasper, C., Rasmussen, H., Kastrup, J. S., Ikemizu, S., Jones, E. Y., Berezin, V., Bock, E., and Larsen, I. K. Nat. Struct. Biol 2000 May.; 7(5):389.-93. 7, 389-393.
Kiselyov, V. V., Berezin, V., Maar, T. E., Soroka, V., Edvardsen, K., Schousboe, A., and Bock, E. (1997) Journal of Biological Chemistry 272, 10125-10134.
Kolkova, K., Novitskaya, V., Pedersen, N., Berezin, V., and Bock, E. J Neurosci 2000 Mar. 15; 20(6):2238.-46 20, 2238-2246.
Krog, L., Olsen, M., Dalseg, A. M., Roth, J., and Bock, E. (1992) Journal of Neurochemistry 59, 838-847.
Lam, K. S., Salmon, S. E., Hersh, E. M., Hruby, V. J., Kazmierski, W. M., and Knapp, R. J. (1991) Nature 354, 82-84.
Maar, T. E., Ronn, L. C. B., Bock, E., Berezin, V., Moran, J., Pasantes-Morales, H., and Schousboe, A. (1997) Journal of Neuroscience Research 47, 163-172.
Milev, P., Maurel, P., Haring, M., Margolis, R. K., and Margolis, R. U. (1996) Journal of Biological Chemistry 271, 15716-15723.
Ranheim, T. S., Edelman, G. M., and Cunningham, B. A. (1996) Proceedings of the National Academy of Sciences of the United States of America 93, 4071-4075.
Rao, Y., Zhao, X., and Siu, C. H. (1994) Journal of Biological Chemistry 269, Schuch et al., 198940-Schuch et al., 198948.
Rasmussen, S., Ramlau, J., Axelsen, N. H., and Bock, E. (1982) Scandinavian Journal of Immunology 15, 179-185.
Rønn, L. C. B., Olsen, M., Østergaard, S., Kiselyov, V. V., Berezin, V., Mortensen, M. T., Lerche, M. H., Jensen, P. H., Soroka, V., Saffell, J. L., Doherty, P., Poulsen, F. M., Bock, E., and Holm, A. (1999) Nature Biotechnology 17, no. 10, 1000-1005.
Rønn, L. C., Ralets, I., Hartz, B., Berezin, V., Beck, M., Berezin, A., Møller, A., and Bock, (2000) E. J. Neurosci. Meth 100, 25-32.
Schmid, R. S., Graff, R. D., Schaller, M. D., Chen, S., Schachner, M., Hemperly, J. J., and Maness, P. F. (1999) Journal of Neurobiology 38, 542-558.
Schuch, U., Lohse, M. J., and Schachner, M. (1989) Neuron 3, 13-20.
Skladchikova, G., Ronn, L. C., Berezin, V., and Bock, E. (1999) Journal of Neuroscience Research 57, 207-218.
Thomsen, N. K., Soroka, V., Jensen, P. H., Berezin, V., Kiselyov, V. V., Bock, E., and Poulsen, F. M. (1996) Nature Structural Biology 3, 581-585.
Williams, E. J., Furness, J., Walsh, F. S., and Doherty, P. (1994) Neuron 13, 583-594.

1-3. (canceled)

4. A peptide compound comprising at least seven amino acid residues, of the formula A-P2-P3-P4-P5-P6-P7, wherein
A is the amino acid residue A;
P2 is selected from the group consisting of the amino acid residues D, E, P, I, Y, V, G, F, Q, N, W, A and T;
P3 is selected from the group consisting of the amino acid residues D, E, I, X, W, V, N, T, G, a hydrophilic amino acid and Y;
P4 is selected from the group consisting of the amino acid residues D, E, W, F, X, T, L, A, G, P, Y and T,
P5 is selected from the group consisting of the amino acid residues D, E, W, A, V, G, N, I and F,
P6 is selected from the group consisting of the amino acid residues D, E, F, S, W, Q, Y and I,
P7 is selected from the group consisting of the amino acid residues D, E, T, S, W, Q, I, V, P, L, N and F.

5. The compound according to claim 4, wherein P2 is selected from the group consisting of the amino acid residues D, E, P, I, Y, V, G, Q, F, W and T.

6. The compound according to claim 4, wherein P2 is selected from the group consisting of the amino acid residues D, E, P, I, Y, V, N, G, F, W and T.

7. The compound according to claim 4, wherein P2 is selected from the group consisting of the amino acid residues D, E, P, I, Y, V, N, F, Q, W and T.

8. The compound according to claim 4, wherein P2 is selected from the group consisting of the amino acid residues D, E, P, I, Y, V, N, F, G, Q and W.

9. The compound according to claim 4, wherein P2 is selected from the group consisting of the amino acid residues D, E, P, I, V, F and W.

10. The compound according to claim 4, wherein P3 is selected from the group consisting of the amino acid residues D, E, I, W, V and N.

11. The compound according to claim 4, wherein P3 is selected from the group consisting of the amino acid residues D, E, I, W, V and T.

12. The compound according to claim 4, wherein P3 is selected from the group consisting of the amino acid residues D, E, V or I.

13. The compound according to claim 4, wherein P4 is selected from the group consisting of the amino acid residues D, E, W, F, A, G, P, Y and T.

14. The compound according to claim 4, wherein P4 is selected from the group consisting of the amino acid residues D, E, W, L, A, G, P, Y and T.

15. The compound according to claim 4, wherein P4 is selected from the group consisting of the amino acid residues D, E, W, F, L, A, G, P and Y.

16. The compound according to claim 4, wherein P4 is selected from the group consisting of the amino acid residues D, E, W, F and P.

17. The compound according to claim 4, wherein P4 is selected from the group consisting of the amino acid residues D, E and T.

18. The compound according to claim 4, wherein P5 is selected from the group consisting of the amino acid residues D, E, W, A, V, E, N, I and F.

19. The compound according to claim 4, wherein P5 is selected from the group consisting of the amino acid residues D, E, W, A, V, G, E, I and F.

20. The compound according to claim 4, wherein P5 is selected from the group consisting of the amino acid residues D, E, W, V, I and F.

21. The compound according to claim 4, wherein P6 is selected from the group consisting of the amino acid residues D, E, F, W, Q, Y and I.

22. The compound according to claim 4, wherein P6 is selected from the group consisting of the amino acid residues D, E, F, S, W, Y and I.

23. The compound according to claim 4, wherein P6 is selected from the group consisting of the amino acid residues D, E, W and Q.

24. The compound according to claim 4, wherein P6 is selected from the group consisting of the amino acid residues D, E and T.

25. The compound according to claim 4, wherein P7 is selected from the group consisting of the amino acid residues D, E, T, S, W, I, V, P, L and F.

26. The compound according to claim 4, wherein P7 is selected from the group consisting of the amino acid residues D, E, T, Q, W, I, V, P, L and F.

27. The compound according to claim 4, wherein P7 is selected from the group consisting of the amino acid residues D, E, S, W, Q, I, V, P and F.

28. The compound according to claim 4 wherein
P2 is selected from the group consisting of the amino acid residues D, E, P, I, G, N, Q, A and V,
P3 is selected from the group consisting of the amino acid residues D, E, I, W, V, N and T,
P4 is selected from the group consisting of the amino acid residues D, E, T and W,
P5 is selected from the group consisting of the amino acid residues D, E, W, V, I and F,
P6 is selected from the group consisting of the amino acid residues D, E, Q and W, and
P7 is selected from the group consisting of the amino acid residues S, W, Q, I, V, P and F.

29. A compound comprising at least seven amino acid residues,
of the formula A-P2-P3-P4-P5-P6-P7-P8-P9, wherein
A is the amino acid residue A
P2 is one amino acid selected from the group consisting of the amino acid residues K, D, A, L, P, G, and F, or a bond,
P3 is one amino acid selected from the group consisting of the amino acid residues K, W, R, Y, L, N, H, and V, I and S, or a bond,
P4 is one amino acid selected from the group consisting of the amino acid residues K, M, Y, T, F, I, N, S, and H and P, or a bond,
P5 is one amino acid selected from the group consisting of the amino acid residues W, E, K, N, F, S, Y, V, D, Q, and A, or a bond,
P6 is one amino acid selected from the group consisting of the amino acid residues K, A, N, M, F, Q, L, V, Y, and W, or a bond,
P7 is one amino acid selected from the group consisting of the amino acid residues K, S, P, W, Y, I, A, L, V, F, and M, or a bond,
P8 is one amino acid selected from the group consisting of the amino acid residues K, T, W, P, N, Y, S, V, I, G, A, F, L, and M, or a bond,
P9 is selected from the group consisting of the amino acid residues W, K, D, S, V, G, A, F, M, Q, and L, or a bond.

30. The compound according to claim 29, wherein P2 is selected from the group consisting of the amino acid residues P, L, K, and A.

31. The compound according to claim 29, wherein P2 is selected from the group consisting of K, A, L, P, A, F, D, and G.

32. The compound according to claim 29, wherein P2 is selected from the group consisting of D and K.

33. The compound according to claim 29, wherein P3 is selected from the group consisting of K, W, R, Y, L, H and V.

34. The compound according to claim 29, wherein P3 is selected from the group consisting of the amino acid residues W, L and Y.

35. The compound according to claim 29, wherein P3 is K or R.

36. The compound according to claim 29, wherein P4 is selected from the group consisting of the amino acid residues K, M, Y, T, F, I, S and H.

37. The compound according to claim 29, wherein P4 is selected from the group consisting of the amino acid residues K, M, Y, F, I, N, S and H.

38. The compound according to claim 29, wherein P4 is selected from the group consisting of the amino acid residues K, M, Y, T, F, I, N and H.

39. The compound according to claim 29, wherein P4 is selected from the group consisting of the amino acid residues M, F and I.

40. The compound according to claim 29, wherein P4 is K.

41. The compound according to claim 29, wherein P5 is selected from the group consisting of the amino acid residues W, E, K, N, F, S, Y, V, D and A.

42. The compound according to claim 29, wherein P5 is selected from the group consisting of the amino acid residues W, K, N, F, S, Y, V, D, Q and A.

43. The compound according to claim 29, wherein P5 is selected from the group consisting of the amino acid residues W, E, K, N, F, S, Y, V, Q and A.

44. The compound according to claim 29, wherein P5 is selected from the group consisting of the amino acid residues W, F, V or Y.

45. The compound according to claim 29, wherein P5 is K.

46. The compound according to claim 29, wherein P5 is selected from the group consisting of the amino acid residues W, E, K, N, F, S, Y, V, D and A.

47. The compound according to claim 29, wherein P6 is selected from the group consisting of the amino acid residues K, A, N, M, F, L, V, Y and W.

48. The compound according to claim 29, wherein P6 is selected from the group consisting of the amino acid residues K, A, M, F, Q, L, V, Y and W.

49. The compound according to claim 29, wherein P6 is selected from the group consisting of the amino acid residues A, M, F, L, V and W.

50. The compound according to claim 29, wherein P6 is K.

51. The compound according to claim 29, wherein P7 is selected from the group consisting of the amino acid residues K, P, W, Y, I, A, L, V, F and M.

52. The compound according to claim 29, wherein P7 is selected from the group consisting of the amino acid residues P, W, I, A, L, V, F and M, or Y.

53. The compound according to claim 29, wherein P7 is K.

54. The compound according to claim 29, wherein P8 is selected from the group consisting of the amino acid residues T, W, P, N, Y, S, V, I, G, A, F, L and M.

55. The compound according to claim 29, wherein P8 is selected from the group consisting of the amino acid residues W, P, V, I, A, F, L and M.

56. The compound according to claim 29, wherein P8 is selected from the group consisting of the amino acid residues T, N and Y.

57. The compound according to claim 56, wherein P8 is Y.

58. The compound according to claim 29, wherein P8 is selected from the group consisting of the amino acid residues the group consisting of W, P, V, I, A, F and L or Y.

59. The compound according to claim 29, wherein P9 is selected from the group consisting of the amino acid residues W, K, D, S, V, G, A, F, M, Q and L.

60. The compound according to claim 29, wherein P9 is selected from the group consisting of the amino acid residues W, V, A, F, M and L.

61. The compound according to claim 29, wherein P9 is selected from the group consisting of the amino acid residues N, Y and S.

62. The compound according to claim 61, wherein P9 is Y.

63. The compound according to claim 29, wherein P9 is selected from the group consisting of the amino acid residues the group consisting of K, D, S and G.

64. The compound according to claim 29, wherein P9 is K.

65. (canceled)

66. The compound according to claim 4, capable of binding to the NCAM Ig2 domain.

67. The compound according to claim 87, capable of binding to the homophilic binding site of the Ig1-Ig2 domains which is constituted by the Ig2 domain.

68. The compound according to claim 4, capable of binding to the NCAM Ig1 domain.

69. The compound according to claim 66, capable of binding to the homophilic binding site of the Ig1-Ig2 domains which is constituted by the Ig1 domain.

70. The compound according to claim 4, capable of binding to the NCAM Ig3 domain.

71. The compound according to claim 70, capable of binding to the homophilic binding site of the Ig3-Ig4 domains which is constituted by the Ig3 domain.

72. The compound according to claim 4, capable of binding to the NCAM Ig4 domain.

73. The compound according to claim 72, capable of binding to the heterophilic binding site of the Ig4 domain.

74. The compound according to caim 4, capable of binding to the NCAM Ig5 domain.

75. The compound according to claim 74, capable of binding to the heterophilic binding site of the Ig5 domain.

76. The compound according to claim 4, capable of binding to the NCAM FN3,1 domain.

77. The compound according to claim 76, capable of binding to the heterophilic binding site of the NCAM FN3,1.

78. The compound according to claim 4, capable of binding to the NCAM FN3,2 domain.

79. The compound according to claim 78, capable of binding to the homophilic binding site of the NCAM FN3,2 domain.

80. The compound according to claim 4, capable of binding to the NCAM Ig1 domain through a binding motif which comprises at least 2 basic amino acid residues.

81. The compound of claim 780, comprising at least 2 basic amino acid residues within a sequence of 10 amino acid residues.

82. The compound according to claim 4, capable of binding to the NCAM Ig2 binding site on the NCAM Ig1 domain.

83. The compound according to claim 4, capable of binding to a binding site on the NCAM Ig1 domain, wherein the binding site is different from the NCAM Ig2 binding site.

84-86. (canceled)

87. The compound according to claim 1, wherein the peptide comprises SEQ ID NO: 1 or SEQ ID NO:2 or SEQ ID NO:3 or SEQ ID NO:4 or SEQ ID NO:5 or SEQ ID NO:6 or SEQ ID NO:7 or SEQ ID NO:8, or SEQ ID NO:9, or SEQ ID NO:10, or SEQ ID NO:11, or SEQ ID NO:12, or SEQ ID NO:13, or SEQ ID NO:14, or SEQ ID NO:15, or SEQ ID NO:16, or SEQ ID NO:17, or SEQ ID NO:18, or SEQ ID NO:19, or SEQ ID NO:20, or SEQ ID NO:21, or SEQ ID NO:22, or SEQ ID NO:23, or SEQ ID NO:24, or SEQ ID NO:25, or SEQ ID NO:26, or SEQ ID NO:27, or SEQ ID NO:28, or SEQ ID NO:29, or SEQ ID NO:30, or SEQ ID NO:31, or SEQ ID NO:32, or SEQ ID NO:33, or SEQ ID NO:34, or SEQ ID NO:35, or SEQ ID NO:36, or SEQ ID NO:37, or SEQ ID NO:38, or SEQ ID NO:39, or SEQ ID NO:40, or SEQ ID NO:41, or SEQ ID NO:42, or SEQ ID NO:43, or SEQ ID NO:44, or SEQ ID NO:45, or SEQ ID NO:46, or SEQ ID NO:47, or SEQ ID NO:48, or SEQ ID NO:49, or SEQ ID NO:50, or SEQ ID NO:51, or SEQ ID NO:52, or SEQ ID NO:53, or SEQ ID NO:54, or SEQ ID NO:55, or SEQ ID NO:56, or SEQ ID NO:57, or SEQ ID NO:58, or SEQ ID NO:59, or SEQ ID NO:60, or SEQ ID NO:61, or SEQ ID NO:62, or SEQ ID NO:63, or SEQ ID NO:64, or SEQ ID NO:65, or SEQ ID NO:66, or SEQ ID NO:67, or SEQ ID NO:68, or SEQ ID NO:69, or SEQ ID NO:70, or SEQ ID NO:71, or SEQ ID NO:72, or SEQ ID NO:73, or SEQ ID NO:74, or SEQ ID NO:75, or SEQ ID NO:76, or SEQ ID NO:77, or SEQ ID NO:78, or SEQ ID NO:79, or SEQ ID NO:80, or SEQ ID NO:81, or SEQ ID NO:82, or SEQ ID NO:83, or a fragment thereof.

88-90. (canceled)

91. The compound according to claim 4, having a length of not more than 100 amino acid residues.

92. The compound according to claim 4, having a length of not more than 50 amino acid residues.

93. The compound according to claim 4, having a length of not more than 30 amino acid residues.

94. The compound according to claim 4, having a length of not more than 20 amino acid residues.

95. (canceled)

96. The compound according to claim 4, having a length of 8-100 amino acid residues.

97. The compound according to claim 4, having a length of 9-100 amino acid residues.

98. The compound according to claim 4, having a length of 10-100 amino acid residues.

99. The compound according to claim 4, having a binding affinity for NCAM such that the equilibrium constant (Kd) is not more than 10⁻³ M.

100-101. (canceled)

102. A method of stimulating NCAM signalling and/or interfering with cell adhesion which comprises use of an effective amount of a compound of claim 4.

103. (canceled)

104. The method of treating normal, degenerated or damaged NCAM presenting cells which comprises use of an effective amount of a compound of claim 4.

105. The method of claim 104, said compound stimulating differentiation of N-CAM presenting cells and/or survival thereof.

106. The method according to claim 102, comprising treatment of diseases and conditions of the central and peripheral nervous system, or of the muscles.

107. The method according to claim 106, which comprises treatment of diseases or conditions of the central and peripheral nervous system.

108. The method according to claim 106, comprising treatment of postoperative nerve damage, traumatic nerve damage, impaired myelination of nerve fibers, postischaemic condition, Parkinson's disease, Alzheimer's disease, dementias, sclerosis, nerve degeneration associated with diabetes mellitus, disorders affecting the circadian clock or neuro-muscular transmission, schizophrenia, and mood disorders.

109. The method according to claim 106, wherein the compound promotes wound-healing.

110. The method according to claim 102, which comprises treatment of cancer.

111. The method according to claim 102, which comprises preventing cell death of heart muscle cells, such as after acute myocardial infarction, or after angiogenesis.

112. The method according to claim 102, which comprises revascularsation.

113. The method according to claim 102, which comprises stimulation of the ability to learn and/or of the short and/or long term memory.

114. A pharmaceutical composition, comprising a pharmaceutically effective amount of one or more of the compounds of claim 4.

115. The pharmaceutical composition according to claim 114, wherein the compounds are formulated as multimers.

116. The pharmaceutical composition according to claim 114, characterised in that the compounds are dendrimers.

117-119. (canceled)

120. Method of claim 106, wherein the composition is used in combination with a prosthetic device.

121. The method according to claim 120, wherein the device is a prosthetic nerve guide.

122. A prosthetic nerve guide, characterised in that it comprises one or more of the compounds according to claim 4.
